(19) United States
(12) Patent Application Publication
(10) Pub. No.: US 2016/0138108 A1
(43) Pub. Date: May 19, 2016

(54) STROMA BIOMARKERS FOR THE DIAGNOSIS OF PROSTATE CANCER

(71) Applicants: Daniel MERCOLA, Rancho Santa Fe, CA (US); Waldemar LERNHARDT, San Diego, CA (US); Jia ZHENYU, Irvine, CA (US); Michael MCCLELLAND, Carlsbad, CA (US)

(72) Inventors: Daniel MERCOLA, Rancho Santa Fe, CA (US); Waldemar LERNHARDT, San Diego, CA (US); Jia ZHENYU, Irvine, CA (US); Michael MCCLELLAND, Carlsbad, CA (US)

(21) Appl. No.: 14/401,497

(22) PCT Filed: May 15, 2013
(86) PCT No.: PCT/US13/41241

§ 371 Date: Nov. 14, 2014

(51) Int. Cl. C12Q 1/68; G01N 33/574

(52) U.S. Cl. CPC C12Q 1/6886; G01N 33/57434; C12Q 2600/158; G01N 2333/47; G01N 2333/90203; G01N 2333/4703; G01N 2333/78

(57) ABSTRACT

The invention described herein relates in part to compositions, biomarkers and methods for diagnosis and prognosis of prostate lesions, including prostate cancer, including a tissue-based assay providing diagnostic and prognostic information related to prostate cancer. In some embodiments, the invention further relates to improvements in the ability to accurately diagnose prostate cancer, detect early-stage prostate cancer, and localize prostate lesions within the three-dimensional space of the prostate.

### CROSS-REFERENCE TO RELATED APPLICATION

This application claims priority from provisional application 61/647,378 filed 15 May 2012. The contents of the document is incorporated herein by reference.

### STATEMENT OF RIGHTS TO INVENTIONS MADE UNDER FEDERALLY SPONSORED RESEARCH

The U.S. government may have certain rights in this invention.

### TECHNICAL FIELD

The invention described herein relates in part to compositions, biomarkers and methods for diagnosis and prognosis of prostate lesions, including prostate cancer, including a tissue-based assay providing diagnostic and prognostic information related to prostate cancer. In some embodiments, the invention further relates to improvements in the ability to accurately diagnose prostate cancer, detect early-stage prostate cancer, and localize prostate lesions within the three-dimensional space of the prostate.

### BACKGROUND ART

Prostate cancer is still an immense health problem in Europe and North America. According to the American Cancer Society, 11% of the cancer deaths in men are due to prostate cancer. Prostate cancer is the second highest cause of cancer deaths in US behind lung cancer. The US National Cancer Institute estimates that 217,730 new cases of prostate cancer were diagnosed and 32,050 men died from prostate cancer in 2010. The prevalence of prostate cancer in the ageing US population is growing at a rate of 6% annually. Due to the high prevalence rate of prostate cancer, 1 in 5 men will be diagnosed with prostate cancer during their lifetime. Increased attention has been given to prostate cancer screening, diagnostic technology, and available clinical treatment options. Available methods for diagnosing prostate cancer have not been entirely satisfactory, for example, in their accuracy, sensitivity, and/or ability to localize lesions within the prostate. Accordingly, compositions and methods are needed for prostate cancer diagnosis. For example, needed are compositions and methods for accurate and early diagnosis, prognosis, and localization of prostate lesions including prostate tumors. Provided are compositions and methods that address this need.

### SUMMARY OF THE INVENTION

Provided herein are biomarkers, agents, methods, assays, compositions and combinations for diagnostic, prognostic, localization, and predictive methods related to prostate cancer and associated conditions. In one embodiment, the invention provides accurate and early diagnosis, prognosis, and localization of prostate lesions including prostate tumors.

In one embodiment, provided are biomarkers and panels of biomarkers, and agents and methods for detecting the same. The biomarkers include prostate cancer biomarkers and reference biomarkers. In particular, the biomarkers include prostate stroma biomarkers, the expression of which, at one or more locations in the prostate stroma, differs (i.e., is increased or decreased), on its own or as compared to one or more other biomarkers (e.g., relative expression), depending on the presence or absence in the prostate of a growth-dysregulated cell or lesion, e.g., a prostate tumor.

The provided methods include a diagnostic method carried out by contacting a test biological sample, e.g., a test sample from a patient, with an agent that specifically binds to a prostate stroma biomarker and then detecting an amount of binding of the agent or detecting the expression or determining an expression level of the biomarker in the test biological sample. Also provided are agents for use in such methods, and sets of agents, which bind panels of the biomarkers.

In one example, the expression or expression level is detected at one or more particular locations within the prostate. In some aspects, the test biological sample is a non-tumor sample, a non-tumor-bearing sample, a sample that is essentially tumor-free, or does not contain tumor detectable by a standard cancer diagnostic procedure, such as a the well know hematoxylin/eosin stain or biopsies sampled from a patient under TRUS guidance.

In one embodiment, the method detects the presence of a prostate lesion or growth-dysregulated cell in the patient. In one aspect of this embodiment, the test biological sample is a non-tumor sample or is essentially tumor-free. In one embodiment, the sample comprises a fixed prostate tissue sample.

In one embodiment, the test sample is from a prostate biopsy, such as a sample obtained from a needle core biopsy from a prostate.

In some aspects, expression levels are determined by detecting an amount of binding of the agent to the sample, for example, at a particular location within the sample, or generally. In one aspect, the amount of binding indicates an expression level of the prostate stroma biomarker. In another aspect, the method further includes detecting an amount or absence of binding of the agent to another sample, such as a normal or reference sample, or to another location within the same sample, such as another location within a prostate needle biopsy section. In one example of this aspect, the method further includes comparing the amount of binding detected in or the expression level determined for the test biological sample (or particular location therein) to the amount or absence of binding detected in or expression level determined for the reference or normal sample or the other location. In one example, the comparison indicates the presence of prostate lesion or cancer or growth-dysregulated cell in the test biological sample or in the patient. The reference sample can be from the same patient or a different subject. In another aspect, the amount or expression level can be compared to a normal or reference level or amount obtained in a separate study, such as one available in a database or electronically.

Also provided are agents that specifically hybridize to or specifically bind to the biomarkers, and systems, e.g., kits, containing the agents, for detection of the biomarkers, including the prostate stroma biomarkers, for example, for use in the provide methods. In one example, provided is a system comprising agents that bind to or specifically hybridize to at or about or at least at or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 or more biomarkers, such as those biomarkers described herein.

In one embodiment, the agents and methods are capable of detecting the presence of a prostate tumor with a percent volume coverage of greater than 0, 0.1, 0.2, 0.3, 0.4, 0.5, 0.6, 0.7, 0.8, 0.9, 1, 2, 3, 4, 5, 10, 15, 20, 25, 30, 35, 40, 45, 50, 55, 60, 65, 70, 75, 80, 85, 90, 95, 96, 97, 98, 99%. In another embodiment, with the provided methods or agents, the prostate stroma biomarker exhibits proximity-to-tumor dependent expression at a distance of at least at or about or at or about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15 mm, or more, e.g., has a stroma signal of at least at or about or at or about 1, 2, 3, 4, 5, 6, 7, 8, 9, 10, 11, 12, 13, 14, 15 mm, or more.

In one embodiment, the prostate stroma biomarker exhibits increased expression with increased proximity to a prostate tumor. Thus, in one embodiment, the methods and agents detect increased expression levels of the prostate stroma biomarker with increased proximity to a prostate tumor.

In one embodiment, the prostate stroma biomarker exhibits decreased expression with increased proximity to a prostate tumor. Thus, in one embodiment, the methods and agents detect decreased expression levels of the prostate stroma biomarker with increased proximity to a prostate tumor.

In one embodiment, the methods and agents detect a prostate lesion, growth-dysregulated cell, tumor, or associated condition with at least 80, 85, 90, 95, 96, 97, 98, 99, or 100% accuracy. In one embodiment, the methods and agents detects an early-stage prostate cancer. In one embodiment, they diagnose a prostate cancer or lesion in a patient having previously received an ambiguous or negative diagnosis or that would be ambiguously diagnosed or diagnosed as tumor-free using a standard prostate cancer diagnostic method, such as those described herein, e.g., biopsy methods such as hematoxylin & eosin stained tissue sections of TRUS-guided biopsies, or a patient with equivocal pathology diagnosis.

In one embodiment, the methods and agents are capable of detecting prostate cancer or lesion on a first biopsy. Thus, in one aspect, the patient has not previously had a prostate biopsy.

In one embodiment, the methods and agents are useful for localizing cells and lesions within the prostate, such as growth-dysregulated cells, lesions, or prostate tumors, within the three-dimensional space of the patient's prostate. In one aspect, the methods are carried out by contacting a test sample with an agent that specifically binds to a prostate stroma biomarker, determining an amount of binding of the agent to or detecting expression levels at each of a plurality of locations within the sample, and determining a three-dimensional position of the lesion or growth-dysregulated cell in the prostate, based on the amounts so determined.

In one aspect, the method is performed in conjunction with imaging, or with information derived from a prostate map. In one example, the method includes obtaining one or more prostate biopsy samples from a subject and constructing a sample map, wherein the one or more prostate biopsy sample is mapped to the subject's prostate. Thus in some aspects, the methods include detecting an amount of binding of an agent to or determining an expression level of a prostate stromal biomarker at one ore more locations within the test sample, and comparing the results to a sample map. In one example, the binding amount(s) or expression level(s) are plotted to the sample map in order to determine the location of the growth-dysregulated cell.

In one embodiment, the prostate stroma biomarker is selected from the group consisting of ALDH3A2, PDLIM7 COL4A2, HSPB8, and FBN1 gene products. In another embodiment, it is selected from the group consisting of COL4A2, HSPB8, PDLIM7, ALDH3A2, FBN1, CAV1, DMPK, DPYSL3, KCTD17, SVIL, and CRTAC1 gene products. In another embodiment, it is selected from the group consisting of products of the genes listed in Table 3. In another embodiment, the prostate stroma biomarkers include one or more biomarkers selected from among ALDH3A2, PDLIM7 COL4A2, HSPB8, and FBN1 gene products, or at least two, three, four, or five biomarkers selected from this group of gene products. In one example, the prostate stroma biomarkers include ALDH3A2, PDLIM7 COL4A2, HSPB8, and FBN1 gene products. In one example, the biomarkers include a FBN1 gene product; in one example, the biomarkers include an ALDH3A2 gene product; in one example, the biomarkers include FBN1 and ALDH3A2 gene products. In another example, the biomarkers include a PDLIM7 gene product. In one example, the biomarkers include a COL4A2 gene product; in one example, the biomarkers include PDLIM7 and COL4A2 gene products. In another embodiment, the biomarkers include at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 95, 100, or more products of genes listed in Table 3.

In one embodiment, the biomarker is a protein. In another embodiment, the biomarker is a polynucleotide. In one embodiment, the agent is an antibody or fragment thereof that specifically binds to the biomarker. In one example, the antibody is labeled with a detectable marker, such as an immunofluorescently-, chemically-, or radio-labeled antibody. In one embodiment, the agent is a polynucleotide, such as one that specifically hybridizes to the biomarker.

In one embodiment, the method includes detecting or contacting a sample with agents that bind to a plurality of biomarkers, such as a plurality of prostate stroma biomarkers, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 or more prostate stroma biomarkers.

In one embodiment, the expression level or amount of binding is determined for a location within the prostate stroma. In one aspect, the location is tumor-adjacent, tumor-close, or tumor-near. In one aspect, the location is within 2, 3, 4, 5, 6, 7, 8, 9, 10, 11, 12, 13, 14, or 15 mm of a prostate tumor or lesion, or growth-dysregulated cell.

In one aspect, the prostate stroma biomarkers exhibit proximitiy to tumor (PTT)-dependent expression differences, such that the expression level of the biomarker changes depending on distance from a tumor or lesion. In one embodiment, the invention relates to a tissue-based assay that permits a definitive diagnosis of prostate cancer, particularly for early detection and cases where existing histological standards fail to provide a clear diagnosis. In one aspect, the methods are based on prostate stroma biomarkers that are diagnostic for prostate cancer even if no tumor is present in the tissue sample. The assay can be used alone or with other techniques such as immunofluorescence, immunohistochemistry, and/or imaging to provide additional diagnostic and prognostic information. In some embodiments, a benefit of the disclosed invention is that it provides the ability to localize the site of the nascent tumor within the prostate gland, which in turn will allow for focal treatment options. Focal treatment options can be organ-sparing and can reduce side effects, such as impotence and incontinence, which often accompany radical prostatectomies.

### BRIEF DESCRIPTION OF THE DRAWINGS

FIGS. 1(a)-(d) show histograms of tumor percentage for Databases 1-4, respectively, as described in Example 1. Tumor percentage data for (a) and (b) were provided by SPECS pathologists and for (c) and (d) were estimated using CellPred program as described in Example 1. Asterisks mark tumor percentages of potentially misclassified cases in Dataset 1, indicated by CellPred as perhaps actually non-tumor-bearing.

FIG. 2 shows incidence numbers of 339 probe sets obtained by 105-fold permutation procedure for gene selection as described in Example 1. The dashed horizontal line marks the incidence number=50. All probe sets with an incidence of >50 were selected for training using PAM using all 15 normal biopsy and the 13 original minimum tumor-bearing stroma cases.

FIG. 3 shows a heatmap using the 131 classifier to categorize all training cases as described in Example 1, using the 339 basis probe set to categorize the training cases.

FIG. 4 shows a plot of the Principal Component Analysis of training cases using the 131 probe classifier as described in Example 1.

FIG. 5 shows a heatmap of all 364 test samples used in the study described in Example 1 as characterized by the 131 probe classifier as described in Example 1. The numbered bars above the various groups of cases indicate cases corresponding to the numbered case sets in Table 2.

FIG. 6 shows a heatmap comparison between Dakhova et al. and the Diagnostic classifier described in Example 1 (cluster Diagram of cases using 39 overlapping genes).

FIG. 7 shows results of the study described in Example 2, for detection of two biomarkers using antibodies. FIG. 7A shows signals detected after staining sections with anti-COL4A2 (collagen, type IV, alpha 2, Alexa Fluor® 594) and anti-PDLIM7 (PDZ and LIM domain 7 (enigma), Alexa Fluor® 488) antibodies. Nuclei are labeled with DAPI. The slide was scanned as 20× but shown here at low resolution. FIG. 7B is a graph, showing total integrated pixel intensity (Y axis) versus distance from tumor (mm; X-axis) for detection on stroma cells of expression of two biomarkers (COL4A2 and PDLIM7), using antibodies specific for COL4A2 and PDLIM7 staining, demonstrating tumor proximity-dependent fluorescent signal intensity, as described in Example 2.

FIG. 8 shows results obtained using Human Protein Atlas (HPA) images (˜1 mm diameter cores) as described in Examples 2 and 3. Panels A and C show images obtained after immunohistochemical staining and staining for FBN1 and ALDH3A2 biomarkers. Visually-observed expression gradients are shown. Panels B and D show results obtained from automated analysis using a custom imaging algorithm, plotting pixel intensity and differences at fixed distances from tumor (contour lines in panels A and C, with gland/cancer mask marked with black lines at the left-hand side of the ˜1 mm diameter cores).

FIG. 9 shows a schematic representation of TRUS (Transrectal Ultrasound) biopsy (A), and H&E staining of prostate biopsy tissue (B and C).

FIG. 10 shows a schematic representation of the extension of volume coverage using embodiments of the provided methods. The cylinder in panel A represents a hollow needle biopsy core, in an assay in which diagnostic information is confined to the core. With such a method, if the tumor is missed by the needle (as shown in this panel, with the core at least at one point being 3 mm away from the tumor), cancer cannot be diagnosed. Panel B depicts an assay according to a provided embodiment, in which a sample obtained from a needle biopsy (again shown as a cylinder with the same diameter) is stained with one or more stroma biomarkers, having a larger stroma signal, such that the assay has extended “reach” or larger volume coverage (represented by the larger cylinder) than the assay shown in panel A. In this exemplary assay, detection of the tumor is possible even if the needle biopsy core does not contain the tumor lesion, e.g., is tumor-free. Thus, even if the tumor is missed by the needle, the exemplary assay can detect the presence of cancer.

FIG. 11 shows various assessments of percent volume coverage using exemplary embodiments of the provided assay. Panel A shows percent coverage of prostate volume for two examples of stroma signal extension from tumor (3 and 5 mm) for a biopsy core diameter of 0.84 mm and core length of 14 mm. Values are shown for 3 average prostate sizes: Small 27.5 cm³; Medium 35 cm³; and Large 60 cm³. The inset represents coverage of the biopsy core only (standard biopsy); standard biopsy procedure covers 0.67%, 0.53%, and 0.31%, of the Normal, Medium, and Large prostates, respectively. Panel B shows percent coverage of prostate volume (for the same exemplary sized prostates) for various stroma signal distances from tumor. Panel C shows additional volume coverage of prostate provided by stroma signal distance in multiples of the volume coverage of the biopsy core without stroma signal, i.e. 0 mm stroma signal, is 1λ. At 5 mm stroma signal distance, each biopsy core covers 167 times of the prostate volume covered by a standard biopsy core. Panel D shows volume coverage of prostate if there is overlapping of biopsy needle coverage; four indicative examples are shows: no overlap, 20% overlap, 40% overlap, and 60% overlap. The coverage of prostate volume decreases as more needle overlapping with each other's coverage.

FIG. 12 shows a schematic representation of one embodiment of the provided assay. The diagnosis can be made and transmitted electronically to the patient's urologist; the images can provide a permanent record for the patient's electronic chart.

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 2nd. edition (1989) Cold Spring Harbor Laboratory Press, Cold Spring Harbor, N.Y. As appropriate, procedures involving the use of commercially available kits and reagents are generally carried out in accordance with manufacturer defined protocols and/or parameters unless otherwise noted.

As used herein, a “prostate cancer biomarker” refers to a biological molecule, such as a gene product, the expression or presence of which (e.g., the expression level or expression profile) on its own or as compared to one or more other biomarkers (e.g., relative expression) differs (i.e., is increased or decreased) depending on the presence, absence, type, class, severity, metastasis, location, stage, prognosis, associated symptom, outcome, risk, likelihood of treatment responsiveness, predicted survival, or prognosis of a prostate cancer or prostate cancer patient, or is associated positively or negatively with such factors or the prediction thereof. In some examples, the prostate cancer biomarkers are biomarkers that are expressed within a prostate tumor or lesion and/or those whose expression differs within a prostate tumor, lesion, or tumor cell, as compared with non-tumor tissues and cells from the same subject or other subject. In other examples, the prostate cancer biomarkers are prostate stroma biomarkers.

As used herein, “prostate stroma biomarker” refers to a biological molecule, such as a gene product, whose expression at one or more locations in the prostate stroma differs (i.e., is increased or decreased), on its own or as compared to one or more other biomarkers (e.g., relative expression), depending on the presence or absence in the prostate of a growth-dysregulated cell or lesion, e.g., a prostate tumor. Typically, the prostate stroma biomarkers exhibit proximity to tumor (PTT)-dependent expression differences, such that the expression level of the biomarker changes depending on distance from a tumor or lesion.

“Stroma signal” refers to the proximity-to-tumor-dependent differences in expression levels observed for the provided stromal biomarkers. A particular distance of “stroma signal” (e.g., 8 mm stroma signal) refers to the distance from a tumor or lesion at which there is a detectable difference in expression levels, e.g., a PTT-dependent difference in expression levels, of a stroma biomarker. For a given stroma biomarker, the stromal signal can vary, for example, depending on the assay used to detect expression levels of the biomarkers. In some embodiments of the provided methods and agents, the biomarker exhibit at or about or least at or about 1, 2, 3, 4, 5, 6, 7, 8, 9, 10, 11, 12, 13, 14, 15 mm, or greater stroma signal, e.g., at least at or about 1, 2, 3, 4, 5, 6, 7, 8, 9, 10, 11, 12, 13, 14, 15 mm, or greater PTT-dependent stromal signal.

As used herein, when describing a method or agent for diagnosis of prostate lesions, prostate growth-dysregulated cells, or prostate cancer, “volume coverage” refers to the volume (e.g., cm³) of prostate tissue over which the method or agent is capable of detecting a tumor, growth-dysregulated cell, or lesion. For example, a method with a volume coverage of 25 cm² is capable of detecting a tumor within a 25 cm² (25 mL) region of the prostate. In some examples, a percent volume coverage is given.

As used herein, when describing a method or agent for diagnosis of prostate lesions, prostate growth-dysregulated cells, or prostate cancer, “percent volume coverage” refers to the percentage by volume of the total prostate in which the method or agent is capable of detecting a tumor. For example, for a given prostate volume, a cancer diagnostic method with a 60% percent volume coverage can detect tumors located within 60% of the prostate, but not within the remaining 40%. A method with 100% volume coverage can detect a tumor located anywhere within the prostate. For a given assay, the “percent volume coverage” will vary, for example, with prostate size. For a method based on analysis of biopsy cores, percent volume coverage will depend, for example, on the number of biopsy core samples analyzed. For example, the percent volume coverage will generally be greater with a 12-core biopsy procedure than for analysis of a single core. In some aspects, the provided methods and agents provide a volume coverage that is at or about or greater than at or about 1, 2, 3, 4, 5, 10, 15, 20, 25, 30, 35, 40, 45, 50, 55, 60, 65, 70, 75, 80, 85, 90, 95, 96, 97, 98, 99% of a prostate.

Volume coverage can also be expressed in terms of a comparison to other assays. For example, in some embodiments, the provided diagnostic assays exhibit improved volume coverage compared to existing methods for prostate cancer diagnosis. For example, in some aspects, the provided methods and agents provide a volume coverage that is at or about or at least at or about 2, 3, 4, 5, 6, 7, 8, 9, 10, or more times that observed with an available method or assay, such as with a standard biopsy method, an assay with a stroma signal of zero, or a method which depends on the detection of expression, expression levels, or differences in expression of a biomarker within a prostate tumor.

Additional definitions are provided throughout the subsections, which follow.

Diagnosis and Prognosis of Prostate Cancer

The presently disclosed invention relates in part to methods, agents, and compositions for the diagnosis and prognosis of prostate cancer. In one embodiment, the invention relates to a tissue-based assay that permits a definitive diagnosis of prostate cancer, particularly for early detection and cases where existing histological standards fail to provide a clear diagnosis. In one aspect, the methods are based on prostate stroma biomarkers that are diagnostic for prostate cancer even if no tumor is present in the tissue sample. The assay can be used alone or with other techniques such as immunofluorescence, immunohistochemistry, and/or imaging to provide additional diagnostic and prognostic information. In some embodiments, a benefit of the disclosed invention is that it provides the ability to localize the site of the nascent tumor within the prostate gland, which in turn will allow for focal treatment options. Focal treatment options can be organ-sparing and can reduce side effects, such as impotence and incontinence, which often accompany radical prostatectomies.

Over one million prostate biopsy procedures are carried out in the U.S. every year (Marks L S, Bostwick D G. Prostate Cancer Specificity of PCA3 Gene Testing: Examples from Clinical Practice. Rev Urol 2008; 10(3):175-81), with over 60% read as negative (O'Dowd G J, Miller M C, Orozco R, Veltri R W. In the US, existing histopathology methods result in ambiguous diagnoses for approximately 300,000 biopsies of the annual 1 million prostate biopsies performed. Thus, approximately 30% of the 1 million biopsies performed per year in the U.S. yield ambiguous results; in addition, it is estimated that approximately 30% of cancers are missed with current technology. False diagnoses result in treatment delay and/or over treatment causing increased unnecessary side effects and healthcare costs. Ambiguous biopsies necessitate re-biopsy after 3-12 months and result in treatment delay, patient anxiety and increased healthcare costs. These limitations indicate large unmet clinical needs and substantial market opportunities for the development and commercialization of highly accurate diagnostic tests, which improve patient outcomes and provide substantial healthcare cost savings.

Analysis of repeated biopsy results within 1 year after a noncancer diagnosis. Urology 2000; 55(4):553-9; Che M, Sakr W, Grignon D. Pathologic features the urologist should expect on a prostate biopsy. Urol Oncol 2003; 21(2):153-61; Pepe P, Aragona F. Saturation prostate needle biopsy and prostate cancer detection at initial and repeat evaluation. Urology 2007; 70(6):1131-5). Even the best available methods, including transrectal ultrasound (TRUS) procedures, can miss up to 30% of clinically significant prostate cancers (Andriole G L, Bullock T L, Belani J S, et al. Is there a better way to biopsy the prostate? Prospects for a novel transrectal systematic biopsy approach. Urology 2007; 70(6 Suppl):22-6).

About, 20-30% of patients that are negative on initial biopsy are re-biopsied in ˜3 to ˜12 months (˜190,000 patients owing to the presence of prostatic intraepithelial neoplasia (PIN), high-grade prostatic intraepithelial neoplasia (HGPIN), atypical small acinar proliferation (ASAP) or other grounds for clinical suspicion of the presence of tumor (O'Dowd G J, Miller M C, Orozco R, Veltri R W. Analysis of repeated biopsy results within 1 year after a noncancer diagnosis. Urology 2000; 55(4):553-9; Che M, Sakr W, Grignon D. Pathologic features the urologist should expect on a prostate biopsy. Urol Oncol 2003; 21(2):153-61; Pepe P, Aragona F. Saturation prostate needle biopsy and prostate cancer detection at initial and repeat evaluation. Urology 2007; 70(6):1131-5; Mian B M, Naya Y, Okihara K, Vakar-Lopez F, Troncoso P, Babaian R J. Predictors of cancer in repeat extended multisite prostate biopsy in men with previous negative extended multisite biopsy. Urology 2002; 60(5):836-40; Leite K R, Camara-Lopes L H, Cury J, Dall'oglio M F, Sanudo A, Srougi M. Prostate cancer detection at rebiopsy after an initial benign diagnosis: results using sextant extended prostate biopsy. Clinics 2008; 63(3):339-42).

Many repeat biopsies are found to be adenocarcinoma. For example, 16-23% of HGPIN and up to 59% of ASAP cases prove to be adenocarcinoma upon repeat biopsy (O'Dowd G J, Miller M C, Orozco R, Veltri R W. Analysis of repeated biopsy results within 1 year after a noncancer diagnosis. Urology 2000; 55(4):553-9; Che M, Sakr W, Grignon D. Pathologic features the urologist should expect on a prostate biopsy. Urol Oncol 2003; 21(2):153-61; Pepe P, Aragona F. Saturation prostate needle biopsy and prostate cancer detection at initial and repeat evaluation. Urology 2007; 70(6):1131-5; Amin M M, Jeyaganth S, Fahmy N, et al. Subsequent prostate cancer detection in patients with prostatic intraepithelial neoplasia or atypical small acinar proliferation. Can Urol Assoc J 2007; 1(3):245-9). Patients deferred to repeat biopsy receive little treatment or guidance during the interim, during which tumors may continue to progress. Methods are needed to resolve false negative and equivocal cases.

Improved diagnostic technology and commercially available diagnostic assays are needed to provide physicians and patients with better means to establish an earlier definitive diagnosis for ambiguous cases. Among the provided embodiments is a robust clinical diagnostic assay that reduces the number of false negative and false positive diagnoses and improves diagnostic capabilities for ambiguous cases.

Available Screening and Diagnostic Methods and Limitations

The American Cancer Society (ACS) recommends men age 50 and above to undergo screening for prostate cancer. The American Urological Association (AUA) revised its guidance in 2009 and lowered the age where screening should be considered to age 40 for men with a family history of prostate cancer. Screening generally includes few tests: a digital rectal exam (DRE), which detects abnormal anatomical prostate appearance, a blood test that measures prostate specific antigen (PSA) levels (values above 4 ng/mL are considered suspicious), and a PSA velocity, rate of PSA level change, i.e. a value greater than 0.4-0.75 ng/ml/year likely signify a growing cancer. Based on the results of the aforementioned tests and additional clinical findings, such as family history for cancer, Urologists then make individualized decisions whether patients should undergo a biopsy procedure for a diagnosis of cancer.

Transrectal ultrasound (TRUS)-guided needle biopsy is commonly used to retrieve prostate tissue for histological analysis as shown in FIG. 9A. The tissue cores are fixed in formalin and embedded in paraffin (FFPE), stained with hematoxylin & eosin (H&E) and microscopically examined by a pathologist who notes the result as a Gleason score based on pattern of growth and cytological appearance of the observed cancer (FIG. 9). Though H&E (hematoxylin and eosin) staining works well for well-developed cancer lesions (FIG. 9C), this method does not perform well for a large number of men who require more definitive testing following screening of PSA levels and other clinical signs for pre-cancerous lesions (FIG. 9B).

Tumors are rated on a scale from 4-10 where cases with a very low score have a good prognosis and patients with a very high score, such as 9 and 10, have a poor prognosis and are likely to relapse even if the cancer is found to be organ-confined and removed by surgery, treated by aggressive radiation or other treatment methods. Eighty percent of all cases, however, are between the range where the Gleason scores are not reliably predictive. Better methods for prediction of outcome are clearly needed. A clinical test, Post-Op Px (previously known as Prostate Px), released by Aureon Biosciences (Yonkers, N.Y.) attempts to improve prediction of outcome and it appears to compare favorably with existing “nomograms” used for prediction.

Improved diagnostic technology and diagnostic assays are needed, for example, those which provide physicians and patients with better means to establish an earlier definitive diagnosis for ambiguous cases. Methods are needed for personalized diagnostic standards that will lead to better clinical treatment decisions and outcomes for the individual patient. Embodiments provided herein address this need.

The current diagnostic regimen for men suspected of having prostate cancer suffers from three major deficiencies and represent a significant unmet clinical need and thus a substantial market opportunity: 1) Ambiguous first biopsy results, 2) False negative biopsy results, and 3) Difficulty of locating cancer lesions within the prostate gland.

Ambiguous First Biopsies:

Approximately 300,000 of the 1 million new (or initial) biopsies performed annually in the US are diagnosed to be ambiguous, i.e. a definitive diagnosis with regard to the presence of cancer cannot be reached with currently available methods, e.g. H&E staining of FFPE tissue and microscopic viewing by a Pathologist. Table A illustrates the problem: a large number of cases with findings of precancerous lesions present with cancer upon re-biopsy 6-12 months later.

TABLE A
Percentage of Patient Detected with Cancer after Re-Biopsy

| First Biopsy | Cancer detected after re-biopsy |
|---|---|
| Benign prostate tissue | 19% |
| HGPIN | 32% |
| ASAP suspicious for malignancy | 41% |
| ASAP suspicious for malignancy + HGPIN | 53% |

False Negative Biopsies:

Twelve-core biopsies and even saturation biopsies of 18 or more cores, can still miss tumor lesions. It is estimated that as many as 30% of biopsies are false negatives. Andriole et al described a simulated biopsy study that illustrates the problem: biopsies were performed on surgically removed prostate specimens (following radical prostatectomies) with a precise radial distribution and the histology results were compared to surgical pathology results of the same prostatectomy specimen. Even in this controlled study 4/20 (20%) were missed. In the clinical setting the problem is likely to be further confounded: biopsy spacing is not evenly distributed throughout the gland caused by patient movement, involuntary movement of the prostate gland, and the desire to avoid the urethra. Therefore, the false negative rate of 30% as suspected by Andriole et al [1] is probably realistic.

The issue of missing tumors has gained increased urgency due to the revision of the American Urological Association Guidelines in 2009: the age for prostate cancer screening was lowered to age 40 if family history and other clinical factors for cancer exist. The lesions in younger men are likely to be small but tend to be more aggressively growing. While the number of such patients may be low, early detection is especially important for this group to arrive at rapid treatment decisions due to the tendency of this group having an aggressive form of prostate cancer.

Difficulty Determining Location of Lesions:

Current biopsy technology does not routinely allow precise determination of the localization of cancerous lesions. The ability to determine the location of cancerous lesions is important because more informed treatment decisions might be reached if the information regarding the size and the location of lesions within the prostate gland is available.

Possible localized treatment options are emerging including needle-directed therapy methods such as cryotherapy, high-intensity focused ultrasound (HIFU) and photodynamic therapy, various radiation therapies, and emerging microwave- and laser-based therapies. The default therapies are the surgical options, such as open, laparoscopic, or robotic surgery.

Biomarkers, Compositions, and Methods for Diagnosis and Prognosis of Prostate Cancer

Among the provided embodiments are biomarkers, e.g., predictive biomarkers, for the detection, diagnosis, prognosis, and localization of prostate lesions, including prostate cancer, as well as methods and agents for detecting the biomarkers.

The provided biomarkers include gene products, such as DNA, RNA, e.g., transcripts, and protein. Among the provided biomarkers are prostate cancer markers, including prostate stroma biomarkers, the expression of which differs (i.e. is increased or decreased) at one or more locations within the prostate stroma, either on its own or as compared to one or more other biomarkers (e.g., relative expression), depending on the presence or absence of a prostate growth-dysregulated cell, tissue, or lesion, e.g., a prostate tumor.

Typically, the stroma biomarkers exhibit expression changes that depend on proximity to tumor (PTT). Stroma signal refers to the proximity-to-tumor-dependent differences in expression levels observed for the provided stromal biomarkers. A particular distance of “stroma signal” (e.g., 8 mm stroma signal) refers to the distance from a tumor or lesion at which there is a detectable difference in expression levels, e.g., a PTT-dependent difference in expression levels, of a stroma biomarker, for example, using a particular assay as provided herein. For a given stroma biomarker, the stromal signal can vary, for example, depending on the assay used to detect expression levels of the biomarkers. In some embodiments of the provided methods and agents, the biomarker exhibit at or about or least at or about 1, 2, 3, 4, 5, 6, 7, 8, 9, 10, 11, 12, 13, 14, 15 mm, or greater stroma signal, e.g., at least at or about 1, 2, 3, 4, 5, 6, 7, 8, 9, 10, 11, 12, 13, 14, 15 mm, or greater PTT-dependent stromal signal.

In one aspect, the provided methods and compositions detect panels of biomarkers, including two or more stroma biomarkers, such as at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 or more biomarkers, such as those biomarkers described herein.

In some embodiments, the provided methods and compositions also detect “housekeeping,” or reference genes, for example, genes for which differences in expression is known or not expected to correlate with differences in the variables analyzed, for example, with the presence or absence of prostate cancer or with survival time, stage, grade, or other prognostic or diagnostic indication thereof. In some aspects, expression levels of such housekeeping genes are detected and used as an overall expression level standards, such as to normalize expression data obtained for stroma biomarkers or prostate cancer biomarkers across various samples. Housekeeping genes are well known. Examples include 18S rRNA and HPRT1.

The stroma-based biomarkers provide an excellent platform for the development of a tissue-based diagnostic assay. Thus, in another embodiment, provided is a diagnostic tissue-based assay. The invention is based in part on development of a prostate cancer gene expression signature, validated as described in Example 1 on three independent patient cohorts involving 364 prostate cancer cases yielding a prostate stroma-based cancer signature of 114 genes with overall performance accuracy of 97.2% of detecting prostate cancer.

Equivocal and negative biopsies are deficient in diagnostic tumor but contain ample stroma. Stroma is defined as tissue surrounding the tumors. The present invention is based in part on the discovery that stroma near tumor contains certain changes in gene expression, not observed in non-tumor samples. In one embodiment, such proximity-to-tumor (PTT)-dependent changes in gene expression are the basis for the provided diagnostic methods. Stroma signal refers to the proximity-to-tumor-dependent differences in expression levels observed for the provided stromal biomarkers; when referring to a distance (e.g., 8 mm), stroma signal describes the distance from a tumor or lesion at which a particular stroma biomarker exhibits a detectable change in expression levels, for example, using a particular assay as provided herein. Thus, in one aspect, the present invention addresses the problem of ambiguous diagnosis with a stroma-based diagnostic assay that can detect the presence of cancer in such cases. In this aspect, the diagnostic assay leads to earlier diagnosis and prevents repeat biopsies, which can significantly reduce associated healthcare costs and diminish stress and uncertainty for the patient.

Epithelial cells of prostate cancer infiltrate and propagate in a microenvironment consisting largely of myofibroblast cells as well as inflammatory cells and other supporting cells and structures. This mesenchymal component is not passive but responds to signals from the tumor component and in turn alters tumor properties, some of which are essential for tumor growth and progression (Cunha G R, Hayward S W, Wang Y Z. Role of stroma in carcinogenesis of the prostate. Differentiation 2002; 70(9-10):473-85; Cunha G R, Hayward S W, Wang Y Z, Ricke W A. Role of the stromal microenvironment in carcinogenesis of the prostate. Int J Cancer 2003; 107(1):1-10). Stroma plays an important role in cancer progression, including prostate cancer progression. Mouse model studies showed that survival and growth of immortalized non-tumorigenic human prostate epithelial cells as renal 4 subcapsular xenografts required stroma from tumor-bearing prostate (Cunha G R, Hayward S W, Wang Y Z, Ricke W A. Role of the stromal microenvironment in carcinogenesis of the prostate. Int J Cancer 2003; 107(1):1-10). Gene expression changes have been observed at the RNA level specific to the tumor microenvironment of prostate cancer (see, e.g., Ernst T, Hergenhahn M, Kenzelmann M, et al. Decrease and gain of gene expression are equally discriminatory markers for prostate carcinoma: a gene expression analysis on total and microdissected prostate tissue. Am J Pathol 2002; 160(6):2169-80; Tuxhorn J A, Ayala G E, Smith M J, Smith V C, Dang T D, Rowley D R. Reactive stroma in human prostate cancer: induction of myofibroblast phenotype and extracellular matrix remodeling. Clin Cancer Res 2002; 8(9):2912-23; Chandran U R, Dhir R, Ma C, Michalopoulos G, Becich M, Gilbertson J. Differences in gene expression in prostate cancer, normal appearing prostate tissue adjacent to cancer and prostate tissue from cancer free organ donors. BMC Cancer 2005; 5(1):45; Yang S Z, Dong J H, Li K, Zhang Y, Zhu J. Detection of AFPmRNA and melanoma antigen gene-1mRNA as markers of disseminated hepatocellular carcinoma cells in blood. Hepatobiliary Pancreat Dis Int 2005; 4(2):227-33; Verona E V, Elkahloun A G, Yang J, Bandyopadhyay A, Yeh I T, Sun L Z. Transforming growth factor-beta signaling in prostate stromal cells supports prostate carcinoma growth by upregulating stromal genes related to tissue remodeling. Cancer Res 2007; 67(12):5737-46; Richardson A M, Woodson K, Wang Y, et al. Global expression analysis of prostate cancer-associated stroma and epithelia. Diagn Mol Pathol 2007; 16(4):189-97; Dakhova O, Ozen M, Creighton C J, et al. Global gene expression analysis of reactive stroma in prostate cancer. Clin Cancer Res 2009; 15(12):3979-89; van der Heul-Nieuwenhuijsen L, Dits N, Van Ijcken W, de Lange D, Jenster G. The FOXF2 pathway in the human prostate stroma. Prostate 2009)).

Similarly, a variety of protein expression changes have been associated with the microenvironment of prostate cancer. For example, reactive stroma, which is believed to occur in a subset of aggressive tumors, has been shown to correlate with changes in a variety of proteins including FGF2, CTGF, Vimentin, ACTA, COL1A, and Tenascin, some of which have been attributed to epithelial-derived TGFβ (Yang F, Tuxhorn J A, Ressler S J, McAlhany S J, Dang T D, Rowley D R. Stromal expression of connective tissue growth factor promotes angiogenesis and prostate cancer tumorigenesis. Cancer Res 2005; 65(19):8887-959; Tuxhorn J A, Ayala G E, Smith M J, Smith V C, Dang T D, Rowley D R. Reactive stroma in human prostate cancer: induction of myofibroblast phenotype and extracellular matrix remodeling. Clin Cancer Res 2002; 8(9):2912-23).

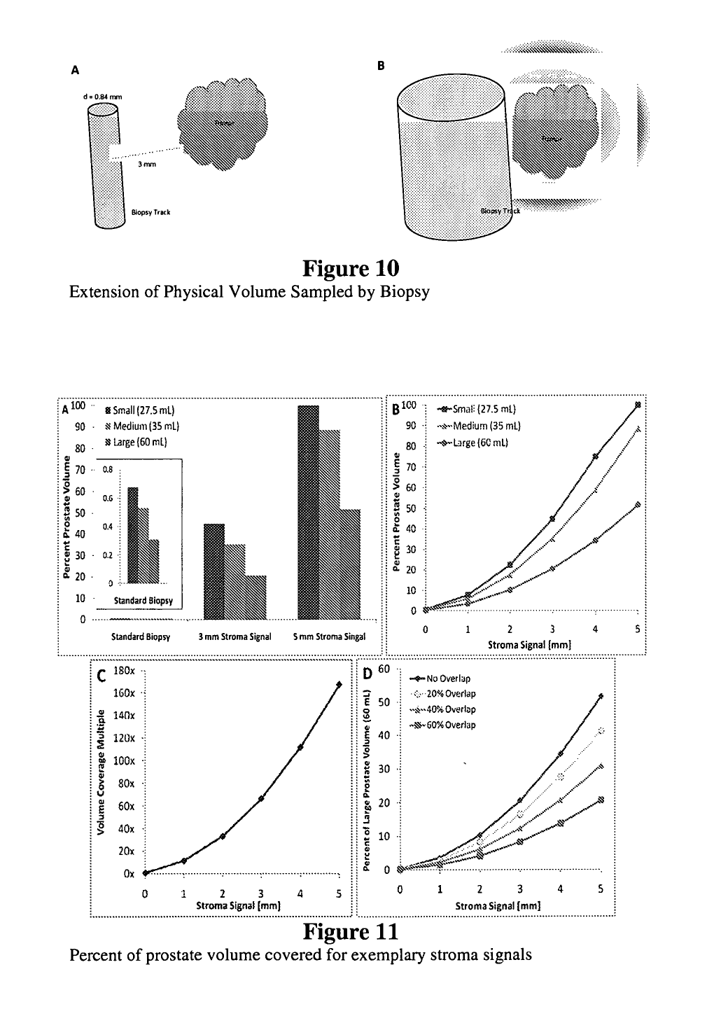

In one embodiment, the provided compositions and methods reduce false negative biopsies by providing increased diagnostic reach by exploiting changes in biomarker expression in stroma surrounding cancerous lesions. In some embodiments, such as that shown in FIG. 10, the provided methods are capable of detecting diagnosing the presence of growth dysregulated cells and lesions in the prostate (e.g., prostate tumor) in a sample, such as that obtained from a biopsy core (e.g., biopsy needle core), that does not contain any actual tumor (“non-tumor bearing” or “tumor-free” sample). By contrast, existing standard methods will not detect cancer if tumor is not retrieved. This additional “reach” of the provided methods and biomarkers is based on the additional volume coverage of stroma signal.

FIG. 10 shows a schematic representation of the extension of volume coverage using embodiments of the provided methods. The cylinder in panel A represents a hollow needle biopsy core, in an assay in which diagnostic information is confined to the core. With such a method, if the tumor is missed by the needle (as shown in this panel, with the core at least at one point being 3 mm away from the tumor), cancer cannot be diagnosed. Panel B depicts an assay according to a provided embodiment, in which a sample obtained from a needle biopsy (again shown as a cylinder with the same diameter) is stained with one or more stroma biomarkers, having a larger stroma signal, such that the assay has extended “reach” or larger volume coverage (represented by the larger cylinder) than the assay shown in panel A. In this exemplary assay, detection of the tumor is possible even if the needle biopsy core does not contain the tumor lesion, e.g., is tumor-free. Thus, even if the tumor is missed by the needle, the exemplary assay can detect the presence of cancer.

The stromal biomarkers typically exhibit “proximity to tumor” (PTT)-dependent changes in expression levels (e.g., fluorescent signal intensity changes detectable using antibody-based assays), or “field effect,” detectable by the provided methods at varying distances away from a prostate tumor. In one aspect, the PTT-dependent changes in expression are detectable in surrounding tissue (stroma), for example, from 1 to 15 mm from a nascent tumor, more specifically, 1, 2, 3, 4, 5, 6, 7, 8, 9, 10, 11, 12, 13, 14, 15 mm distal from the nascent tumor to be detected. In one example, the effect is detectable from at or about 3, 4, 5, 6, 7, or 8 mm from the tumor when applied to prostate cancer tissue. In some aspects, the field effect extends into the stroma, e.g., the tumor-adjacent stroma, tumor-close stroma (˜3 mm), near stroma, intermediate stroma, or far stroma (>15 mm). In one aspect, the effect extends to near, adjacent, close, and/or intermediate stroma but does not extend into far stroma. Thus in one aspect, the provided assay enables the “survey” of a larger fraction of the prostate (greater volume coverage) than possible using existing methods.

For example, the standard biopsy procedure needle has a diameter of 0.84 mm (18 gauge) and the average biopsy tissue length is 14 mm (Vincenzo Ficarra et al., “Needle Core Length is a Quality Indicator of Systematic Transperineal Prostate Biopsy,” European Urology, 50 (2006) 266-271), yielding a total volume of 0.19 cm3 retrieved for the standard 12-core prostate biopsy procedure. The range of the sizes of prostate gland for men between 40 and 70 is between 12 cm3 to 200 cm3. For the purpose of modeling sampling volume coverage, three prostate sizes are selected, 27.5 cm³ (average small), 35 cm³ (average medium), and 60 cm³ (average large) to model the volume coverage of biopsy core needles and extended volume coverage with stroma signals.

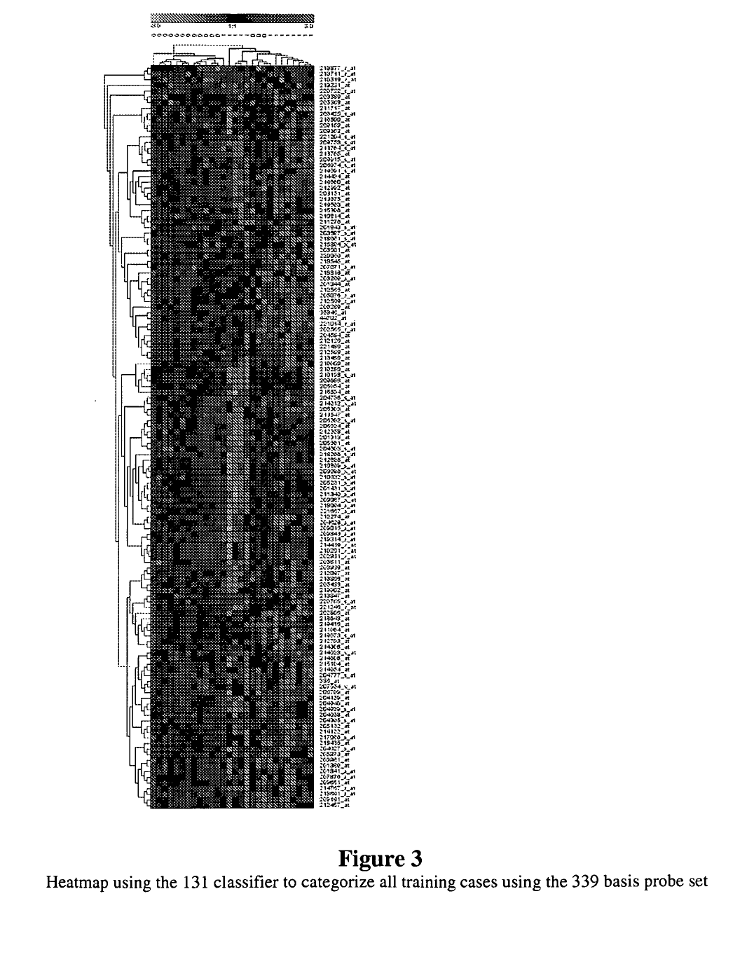

The inset in FIG. 11A shows the corresponding volume coverage of prostate under standard biopsy procedure for the small, medium, and large prostates, at 0.67%, 0.53%, and 0.31%, respectively. FIG. 11A shows the volume coverage of prostate as a function of a modeled stroma signal. A 5 mm stroma signal would provide a theoretical sample volume coverage of 100% for small prostate and 89% and 52%, respectively, for medium and large prostates; these numbers indicate a two orders of magnitude increase in diagnostics and detection power. FIG. 3B illustrates how the volume coverage of model prostate increases as the stroma signal distance increases. FIG. 11C presents the additional volume coverage of prostate provided by stroma signal distance in multiples of the volume coverage of the biopsy core (e.g., biopsy core needle) without stroma signal, i.e. 0 mm stroma signal, is 1×. At 5 mm stroma signal, each biopsy core covers 167 times of the volume covered by a standard biopsy core. However, at this calculated coverage level it must be assumed that potential stroma signals would overlap thus diminishing the covered volume. Nevertheless, significantly more prostate volume would be surveyed than if the diagnostic signal were confined to the needle track alone. Four examples of percent overlap are shown in FIG. 11D, no overlap, 20% overlap, 40% overlap, and 60% overlap assuming an average large prostate volume of 60 mL).

Also provided are methods, compositions, and systems, for the detection of the biomarkers. For example, provided are agents, sets of agents, and systems for detecting the biomarkers and methods for use of the same, such as diagnostic and prognostic uses, e.g., for prostate cancer.

In one embodiment, the agents are proteins (e.g., antibodies), polynucleotides and/or other molecules, which specifically bind to or specifically hybridize to the biomarkers. The agents include polynucleotides, such as probes and primers, e.g. sense and antisense PCR primers, having identity or complementarity to the polynucleotide biomarkers, such as mRNA and/or cDNA; and proteins, such as antibodies, which specifically bind to such biomarkers. Sets and kits containing the agents, such as agents specifically hybridizing to or binding the panel of biomarkers, also are provided.

Thus, the systems, e.g., microarrays, sets of agents polynucleotides, and kits, provided herein include those with proteins (e.g., antibodies) and/or nucleic acid molecules (e.g., DNA oligonucleotides, such as primers and probes, the length of which typically varies between 10 or 15 bases and several kilobases, such as between 20 bases and 1 kilobase, between 40 and 100 bases, and between 50 and 80 nucleotides or between 20 and 80 nucleotides). In one aspect, most (i.e. at least 60% of) nucleic acid molecules of a nucleotide microarray, kit, or other system, are capable of hybridizing to the biomarkers. In some aspects, the agents are labeled with one or more detectable marker, e.g., a fluorescent marker.

In one embodiment, the provided biomarkers, agents, and/or methods are capable of detecting a growth-dysregulated cell or lesion, e.g., tumor with a volume coverage of greater than 1, 2, 3, 4, 5, 10, 15, 20, 25, 30, 35, 40, 45, 50, 55, 60, 65, 70, 75, 80, 85, 90, 95, 96, 97, 98, 99% of a prostate. In other embodiments, the volume coverage is at least 2, 3, 4, 5, 6 or more times that observed with an available method, such as with standard biopsy methods described herein, such as Transrectal ultrasound (TRUS)-guided needle biopsy.

Biological Samples and Sample Acquisition

In some embodiments, expression and expression levels of the biomarkers and housekeeping or reference markers are detected and determined in biological samples, including biological test samples and reference or normal samples. Exemplary biological samples are tissue samples, such as prostate samples, tumor samples, and samples from tissues containing or thought to contain lesions, tumors, or metastases, and fluid samples, such as blood, plasma, serum, stool, urine, saliva, tears, serum and semen samples, and samples prepared from such a tissue or fluid, such as a cell or tissue preparation, including cell suspensions and biopsies, and samples derived from biopsies, such as tissue sections. Typically, the samples are prostate or prostate-derived samples, or samples derived from tissue or fluid thought to contain prostate cancer or a prostate lesion or metastasis thereof.

Such samples may be obtained using any of a number of well-known techniques. In one embodiment, samples are obtained from prostate biopsies, such as from prostate cancer patients, subjects suspected of having prostate cancer or a prostate lesion or being at-risk for such conditions, normal (e.g. healthy) donors, and biopsies obtained during autopsies, such as a rapid autopsy biopsy.

Prostate biopsy samples may be acquired using any of a number of well-known standard techniques. Prostate biopsy techniques include transperineal, transurethral, and transrectal prostate biopsises. In one embodiment, the transrectal prostate biopsy is guided by a transrectal ultrasound (TRUS) through the anus, and into the rectum. In one aspect, utilization of the TRUS assay allows mapping of biological samples obtained during the biopsy to particular regions within the prostate.

Any type of biopsy may be used with the teachings. In one embodiment, the prostate biopsy is obtained in conjunction with one or more imaging methodology, allowing for localization of samples, expression levels, and/or lesions, e.g., tumors, within the three-dimensional space of the prostate. In one aspect, such techniques allow for the exploitation of the prostate microenvironment, for example, to identify and localize the presence of neoplastic cells in a subject's prostate. Such methods can be particularly useful, for example, to inform appropriate focal treatment options, which can be organ-sparing and offer reduced side effects, such as impotence and incontinence, which often accompany radical prostatectomies.

In one embodiment, the prostate biopsy is performed in conjunction with imaging technology and a sample map is produced. In one such aspect, at least two or more sample cores are obtained from a subject's prostate, each creating a track within the prostate; typically, the tracks are mapped to the subject's prostate, such as with 3-dimensional biopsy mapping. In one example, each core, once obtained from the subject, is marked to allow for accurate analysis of the samples and to orient the track within the prostate. Other prostate biopsy methods in conjunction with imaging may be used to produce the sample map.

Diagnostic Assay and Uses of the Provided Biomarkers and Methods

In one embodiment, the provided methods include a tissue-based clinical assay that permits a definitive diagnosis of prostate cancer, for example, in particular for cases where existing histological standards fail to provide a clear diagnosis and/or in early stages of cancer. In one aspect, the test is based on a set of one or more prostate stroma biomarkers that are diagnostic for prostate cancer even if no tumor is present in the tissue sample.

Thus, among the provided embodiments are agents and methods that specifically bind to and detect the biomarkers in a biological sample, for example, to determine the expression levels thereof. In one embodiment, the agents are antibodies that specifically bind to the biomarkers. In one example, the antibodies bind to prostate cancer tissue on FFPE (formalin-fixed and paraffin-embedded) biopsy tissues, tissue samples that are typically used in pathology laboratories for prostate cancer diagnosis.

In some embodiments, the methods and compositions provide accurate diagnosis on the patient's first biopsy and reduce the number of repeat biopsies resulting in decreasing the unnecessary time delay to initiation of cancer treatment. In other embodiments, they provide a substantial reduction in overall healthcare costs, such as by reducing the number of biopsies required for accurate diagnosis, thereby lowering the immediate costs required to reach a diagnosis and prevent the additional costs incurred by side effects, such as bleeding, infection, inflammation, time off from work and other costs that are incurred with the procedure.

In one aspect, the provided diagnostic assays and embodiments are capable of definitive diagnosis of presence or absence of cancer on the patient's first biopsy, which can have the advantage of early detection, diagnosis, and prognosis of small adenocarcinoma lesions and provide physical planning and guidance for potential focal therapies. Of the ˜220,000 diagnosed cases of prostate cancer each year, ˜80% have small and low-grade lesions. For these patients, there is a clear clinical need for adequate and more refined diagnostic tests.

In some embodiments, the assay includes two integrated approaches: a) prostate stroma biomarkers with unique properties diagnostic for “presence of tumor” in tumor-surrounding stroma, even if no tumor is present in biopsy tissue, and b) automated scanning and analysis of biological sample probed with the provided agents, e.g., stained tissue, e.g., with fluorescently labeled antibodies to stroma markers or antibodies to stroma markers visualized by immunochemistry (IHC) to develop a diagnostic assay.

In some embodiments, the assay is used to diagnose patients with newly suspected prostate cancer and to provide physicians with critical information for selecting informed treatment choices for recommendation to patients.

In some embodiments, the assay includes an immunofluorescence (IF) test that combines the use of antibodies to diagnostic prostate cancer stroma biomarkers with high-throughput digital imaging and histocytometry analysis. In one aspect, the assay uses antibody multiplexing, such as by using fluorophores with different emission spectra. For example, the degree of antibody multiplexing can include 2, 3, 4, 5, 6, 7, 8, 9, 10, or more antibodies; in some aspects, the multiplexing allows for inclusion of internal control antibodies.

In one embodiment, the multiplex antibody test is performed on formalin fixed paraffin embedded (FFPE) prostate biopsy tissue, the latter being the standard substrate for prostate cancer diagnosis. In one aspect, both fluorescent and brightfield hematoxylin and eosin (H&E) digital images are obtained, e.g., of the entire tissue section present on the microscope slide, i.e. whole-slide images. This dual output can allow a pathologist to view brightfield and IF images side by side. Fluorescence signal analysis from the diagnostic stroma markers is tested to determine if they indicate the presence of cancer nearby as outlined above. In some aspects, the histopathologist marks “regions of interest” (ROI) on either image. Histocytometry software analyzes the IF image, e.g., the ROI on the IF image, for the presence of cancer based on diagnostic fluorescent marker signal quantization.

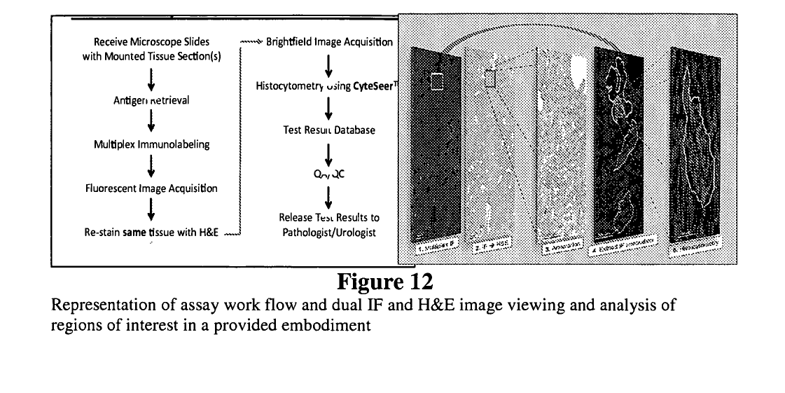

FIG. 12 provides a schematic representation of one embodiment of the provided assay. The diagnosis can be made and transmitted electronically to the patient's urologist; the images can provide a permanent record for the patient's electronic chart.

In some embodiments, the provided biomarkers, agents, and methods are used to diagnose prostate lesions, e.g., to identify “presence-of-tumor,” based solely on the detection of changes in the microenvironment near a focus of tumor by quantitative criteria. In some aspects, the methods are useful in cases of an initial negative biopsies that would otherwise be referred for re-biopsy, owing to the presence of ASAP or PIN. In one example, such determination of “presence of tumor” strengthens guidance for neoadjuvant therapy or prevention therapy or an accelerated scheduling of re-biopsy.

In another example, stroma biomarkers exhibiting expression changes that indicate presence-of-tumor are used as targets for therapeutic intervention. Because stroma facilitates tumor growth, expression changes that occur in stroma indicating the presence-of-tumor might be targets for therapeutic intervention that could leave normal stroma relatively unaffected.

In some embodiments, the methods and compositions are useful for localization of cancer within the prostate gland, and thus are useful in allowing focal treatment options, which can be organ-sparing and allow reduction of side effects, such as impotence and incontinence, which often accompany radical prostatectomies.

Thus, among the provided embodiments are algorithms for the use of biomarkers that show tumor proximity related signal intensity. In one aspect, methods are provided that combine such algorithms with the provided diagnostic assays, generating visualization of vectors for location of the 3-dimensional position of a lesion within the gland. Even with overlapping of stroma signal taken into consideration a large increase in covered volume and therefore diagnostic “reach” is achieved.

Localized treatment options are emerging including needle-directed therapy methods such as cryotherapy, high-intensity focused ultrasound (HIFU) and photodynamic therapy, various radiation therapies, and emerging microwave- and laser-based therapies. The default therapies are the surgical options, such as open, laparoscopic, or robotic surgery.

Exemplary Biomarkers

This section provides structural and functional information for various biomarkers provided herein, including prostate cancer biomarkers, prostate stroma biomarkers, and reference biomarkers. Among the provided biomarkers are prostate cancer biomarkers, prostate stroma biomarkers, and various reference markers, such as those expressed in particular types of tissues or cells, such as epithelial cells and stroma. Such biomarkers include prostate tumor-specific control markers (including AMACR, FOHL1), Epithelial specific control markers (including KRT19, pan-cytokeratin), stroma-specific control (invariant) biomarkers (including ACTA2, CAV1, DES, DPYSL3, FHL1, SVIL, VIM), and Diagnostic prostate stroma biomarkers, the expression levels of which increases (ALDH3A2, PDLIM7) or decreases (COL4A2, HSPB8, FBN1) with distance from a prostate lesion, e.g., tumor. Such exemplary biomarkers and agents (antibodies) for their detection according to the provided methods are summarized in Table B. Table B lists a proposed number of detecting antibodies per type of biomarker, such as 1 to 2 for detection of the tumor-specific control biomarkers, for use in an exemplary multiplexing assay according to embodiments provided herein. Other numbers of agents and biomarkers may be used.

TABLE B

| | | | | Proposed # |
|---|---|---|---|---|
| | | | Exemplary | of Abs |
| Group | Type of Antibodies | | Biomarkers | per Group |
| 1 | Tumor Specific Control | | AMACR, FOLH1 | 1 to 2 |
| 2 | Epithelial Specific Control | | KRT19, pan-cytokeratin | 1 to 2 |
| 3 | Stroma-specific diagnostic Biomarkers, | | ACTA2, CAV1, DES, | 1 to 2 |
| | | | DPYSL3, FHL1, VIM | |
| 4 | Diagnostic Prostate Stroma | Signal Increases with | ALDH3A2, PDLIM7 | 2 |
| | Biomarkers | distance to tumor | | |
| | | Signal decreases with | COL4A2, HSPB8, FBN1 | 2 |
| | | distance from tumor | | |
| 5 | Biomarker with differential | Stroma: | SVIL | 1 |
| | staining patterns between | homogeneous | | |
| | stroma and epithelial tumor | staining; tumor | | |
| | lesions | epithelium: stippled | | |
| | | staining | | |

In one embodiment, the prostate stroma biomarkers include one or more biomarkers selected from among ALDH3A2, PDLIM7 COL4A2, HSPB8, and FBN1 gene products, or at least two, three, four, or five biomarkers selected from this group of gene products. In one example, the prostate stroma biomarkers include ALDH3A2, PDLIM7 COL4A2, HSPB8, and FBN1 gene products. In one example, the biomarkers include a FBN1 gene product; in one example, the biomarkers include an ALDH3A2 gene product; in one example, the biomarkers include FBN1 and ALDH3A2 gene products. In another example, the biomarkers include a PDLIM7 gene product. In one example, the the biomarkers include an COL4A2 gene product; in one example, the biomarkers include PDLIM7 and COL4A2 gene products.

In another embodiment, the biomarkers include at least 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 80, 90, 95, 100, or more products of genes listed in Table 3.

Exemplary Tumor Specific Control Markers

The tumor-specific control markers include AMACR gene products. The AMACR (alpha-methylacyl-CoA racemase) gene encodes a racemase. The encoded enzyme interconverts pristanoyl-CoA and C27-bile acylCoAs between their (R)- and (S)-stereoisomers. The conversion to the (S)-stereoisomers is necessary for degradation of these substrates by peroxisomal beta-oxidation. Encoded proteins from this locus localize to both mitochondria and peroxisomes. Mutations in this gene may be associated with adult-onset sensorimotor neuropathy, pigmentary retinopathy, and adrenomyeloneuropathy due to defects in bile acid synthesis. Alternatively spliced transcript variants have been described. AMACR racemizes 2-methyl-branched fatty acid CoA esters and is responsible for the conversion of pristanoyl-CoA and C27-bile acyl-CoAs to their (S)-stereoisomers and has been suggested for use as a prostate cancer biomarker. AMACR gene products include those described by Zheng et al., “Sequence variants of alpha-methylacyl-CoA racemase are associated with prostate cancer risk,” Cancer Res. 2002 62(22):6485-8; Zha et al., “Alpha-methylacyl-CoA racemase as an androgen-independent growth modifier in prostate cancer,” Cancer Res. 2003 63(21):7365-76; Rubin et al., “Alpha-Methylacyl coenzyme A racemase as a tissue biomarker for prostate cancer,” JAMA 2002 287(13):1662-70; Rubin et al., “Decreased alpha-methylacyl CoA racemase expression in localized prostate cancer is associated with an increased rate of biochemical recurrence and cancer-specific death,” Cancer Epidemiol. Biomarkers Prev. 2005 14(6):1424-32.

The tumor-specific control markers include FOLH1 gene products. The FOLH1 (folate hydrolase (prostate-specific membrane antigen) 1) gene encodes a type II transmembrane glycoprotein belonging to the M28 peptidase family. The protein acts as a glutamate carboxypeptidase on different alternative substrates, including the nutrient folate and the neuropeptide N-acetyl-1-aspartyl-1-glutamate and is expressed in a number of tissues such as prostate, central and peripheral nervous system and kidney. A mutation in this gene may be associated with impaired intestinal absorption of dietary folates, resulting in low blood folate levels and consequent hyperhomocysteinemia. Expression of this protein in the brain may be involved in a number of pathological conditions associated with glutamate excitotoxicity. In the prostate the protein is up-regulated in cancerous cells and is used as an effective diagnostic and prognostic indicator of prostate cancer. This gene likely arose from a duplication event of a nearby chromosomal region. Alternative splicing gives rise to multiple transcript variants encoding several different isoforms. FOLH1 has both folate hydrolase and N-acetylated-alpha-linked-acidic dipeptidase (NAALADase) activity and has a preference for tri-alpha-glutamate peptides. In the intestine, it can be important for the uptake of folate. In the brain, it can modulate excitatory neurotransmission through the hydrolysis of the neuropeptide, N-aceylaspartylglutamate (NAAG), thereby releasing glutamate. Isoform PSM-4 and isoform PSM-5 may not be physiologically relevant. FOLH1 gene products include those described by O'Keefe et al., Mapping, genomic organization and promoter analysis of the human prostate-specific membrane antigen gene. Biochim Biophys Acta. 1998 1443(1-2):113-27; O'Keefe et al., Prostate Specific Membraen Antigen. Prostate cancer: biology, genetics and the new therapeutics 2001 Humana Press. pp. 307-326; O'Keefe et al., Comparative analysis of prostate-specific membrane antigen (PSMA) versus a prostate-specific membrane antigen-like gene. Prostate 2004 58(2):200-10; Perner et al., Prostate-specific membrane antigen expression as a predictor of prostate cancer progression. Hum. Pathol. 2007 38(5):696-701.

The biomarkers include GCPII gene products. This gene, Glutamate carboxypeptidase II (GCPII) (also known as N-acetyl-L-aspartyl-L-glutamate peptidase, NAAG peptidase, or NAALADase) encodes an enzyme that in humans is encoded by the FOLH1 (folate hydrolase (prostate-specific membrane antigen) 1) gene. It was found that there were multiple potential start sites for PSMA as well as multiple alternative splice forms that vary in the type of membrane protein formed or having a cytosolic location and each form probably varies regarding caboxypeptidase activity given the restriction for enzymatic activity for PSMA. PSMA is strongly expressed in the human prostate being a hundredfold greater than the expression in most other tissues. In cancer it is upregulated in expression and has been called the second most up-regulated gene in prostate cancer being increased 8-12 fold over the non cancerous prostate. Because of this high expression it is being developed as a target for therapy and imaging. In human prostate cancer the higher expressing tumors are associated with quicker time to progression and a greater percentage of patients suffering relapse. PSMA is the target of an approved imaging agent for prostate cancer, capromabpentide, PROSTASCINT. Second generation antibodies and low molecular weight ligands for imaging and therapy are being developed.

Epithelial Specific Control Markers

KRT19 (keratin 19): The protein encoded by this gene is a member of the keratin family. The keratins are intermediate filament proteins responsible for the structural integrity of epithelial cells and are subdivided into cytokeratins and hair keratins. The type I cytokeratins consist of acidic proteins which are arranged in pairs of heterotypic keratin chains. Unlike its related family members, this smallest known acidic cytokeratin is not paired with a basic cytokeratin in epithelial cells. It is specifically expressed in the periderm, the transiently superficial layer that envelopes the developing epidermis. The type I cytokeratins are clustered in a region of chromosome 17q12-q21. KRT19 involves in the organization of myofibers. Together with KRT8, helps to link the contractile apparatus to dystrophin at the costameres of striated muscle.

Keratin, type I cytoskeletal 19 also known as cytokeratin-19 (CK-19) or keratin-19 (K19) is a protein that in humans is encoded by the KRT19 gene. Keratin 19 is a type I keratin. Due to its high sensitivity, KRT19 is the most used marker for the RT-PCR-mediated detection of tumor cells disseminated in lymph nodes, peripheral blood, and bone marrow of breast cancer patients. Depending on the assays, KRT19 has been shown to be both a specific and a non-specific marker. False positivity in such KRT19 RT-PCR studies include: illegitimate transcription (expression of small amounts of KRT19 mRNA by tissues in which it has no real physiological role), haematological disorders (KRT19 induction in peripheral blood cells by cytokines and growth factors, which circulate at higher concentrations in inflammatory conditions and neutropenia), the presence of pseudogenes (two KRT19 pseudogenes, KRT19a and KRT19b, have been identified, which have significant sequence homology to KRT19 mRNA. Subsequently, attempts to detect the expression of the authentic KRT19 may result in the detection of either or both of these pseudogenes), sample contamination (introduction of contaminating epithelial cells during peripheral blood sampling for subsequent RT-PCR analysis). Keratin 19 is often used together with keratin 8 and keratin 18 to differentiate cells of epithelial origin from hematopoietic cells in tests that enumerate circulating tumor cells in blood.

KRT19 gene products can be involved in prostate tumor progression. KRT19 products exhibit dipeptidyl-peptidase IV type activity and can cleave Gly-Pro-AMC in vitro.

KRT19gene products include those described by Peehl et al., Keratin 19 in the adult human prostate: tissue and cell culture studies. Cell Tissue Res. 1996 285(1):171-6; Letellier et al., Epithelial phenotypes in the developing human prostate. J Histochem Cytochem. 2007 55(9):885-90; Lacroix, M., Significance, detection and markers of disseminated breast cancer cells. Endocrine-Related Cancer 2006 13(4):1033-67; Walker et al., The intercellular adhesion molecule, cadherin-10, is a marker for human prostate luminal epithelial cells that is not expressed in prostate cancer. Mod Pathol. 2008 21(2):85-95.

The biomarkers include Pan-cytokeratin gene products: Cytokeratins are proteins of keratin-containing intermediate filaments found in the intracytoplasmic cytoskeleton of epithelial tissue. The term “cytokeratin” began to be used in the late 1970s when the protein subunits of keratin intermediate filaments inside cells were first being identified and characterized. In 2006 a new systematic nomenclature for keratins was created and now the proteins previously called “cytokeratins” are simply called keratins. There are two types of cytokeratins: the acidic type I cytokeratins and the basic or neutral type II cytokeratins. Cytokeratins are usually found in pairs comprising a type I cytokeratin and a type II cytokeratin. Basic or neutral cytokeratins include CK1, CK2, CK3, CK4, CK5, CK6, CK7, CK8 and CK9. Acidic cytokeratins are CK10, CK12, CK 13, CK14, CK16, CK17, CK18, CK19 and CK20. The cytokeratins cannot be divided into low versus high molecular weight solely based on their charge. Expression of these cytokeratins is frequently organ or tissue specific. As an example, CK7 is typically expressed in the ductal epithelium of the genitourinary (GU) tract and CK20 most commonly in the gastrointestinal (GI) tract. Histopathologists employ such distinctions to detect the cell of origin of various tumors. The subsets of cytokeratins which an epithelial cell expresses depends mainly on the type of epithelium, the moment in the course of terminal differentiation and the stage of development. Thus this specific cytokeratin fingerprint allows the classification of all epithelia upon their cytokeratin expression profile. Furthermore this applies also to the malignant counterparts of the epithelia (carcinomas), as the cytokeratin profile tends to remain constant when an epithelium undergoes malignant transformation. The main clinical implication is that the study of the cytokeratin profile by immunohistochemistry techniques is a tool of immense value widely used for tumor diagnosis and characterization in surgical pathology. Pan-cytokeratin gene products include those described by Sherwood et al., Differential cytokeratin expression in normal, hyperplastic and malignant epithelial cells from human prostate. J Urol. 1990 143(1):167-71; Yang et al., Rare expression of high-molecular-weight cytokeratin in adenocarcinoma of the prostate gland: a study of 100 cases of metastatic and locally advanced prostate cancer. Am J Surg Pathol. 1999 23(2):147-52; Bassily et al., Coordinate Expression of Cytokeratins 7 and 20 in Prostate Adenocarcinoma and Bladder Urothelial Carcinoma. Am J Clin Pathol. 2000 113(3):383-388; Wolff et al., Cytokeratin 8/18 Levels in Patients with Prostate Cancer and Benign Prostatic Hyperplasia. Urol Int 1998 60(3):152-155; Wolff et al., Cytokeratin markers in patients with prostatic diseases. Anticancer Res. 1999 19(4A):2649-52.

Stroma-Specific Control, Invariant

The biomarkers include gene products of ACTA2 (Alpha-actin-2): A protein encoded by this gene belongs to the actin family of proteins, which are highly conserved proteins that play a role in cell motility, structure and integrity. Alpha, beta and gamma actin isoforms have been identified, with alpha actins being a major constituent of the contractile apparatus, while beta and gamma actins are involved in the regulation of cell motility. This actin is an alpha actin that is found in skeletal muscle. Defects in this gene cause aortic aneurysm familial thoracic type 6. Multiple alternatively spliced variants, encoding the same protein, have been identified. Actins are highly conserved proteins that are involved in various types of cell motility and are ubiquitously expressed in all eukaryotic cells.

Alpha-actin-2 also known as actin, aortic smooth muscle or alpha smooth muscle actin (α-SMA, SMactin, alpha-SM-actin, ASMA) is a protein that in humans is encoded by the ACTA2 gene located on 10q22-q24. Actin alpha 2, the human aortic smooth muscle actin gene, is one of six different actin isoforms which have been identified. Actins are highly conserved proteins that are involved in cell motility, structure and integrity. Alpha actins are a major constituent of the contractile apparatus.

ACTA2 gene products include those described by Leimgruber et al., Dedifferentiation of prostate smooth muscle cells in response to bacterial LPS. Prostate 2010 [Epub ahead of print]; Wang et al., Dedifferentiation of stromal smooth muscle as a factor in prostate carcinogenesis. Differentiation 2002 70(9-10):633-45; Doles et al., Growth, morphogenesis, and differentiation during mouse prostate development in situ, in renal grafts, and in vitro. Prostate 2005 65(4):390-9; Quintar et al., Growth, morphogenesis, and differentiation during mouse prostate development in situ, in renal grafts, and in vitro. Prostate 2010 70(11):1153-65.

The biomarkers include CAV1 (caveolin 1) gene products: A scaffolding protein encoded by this gene is the main component of the caveolae plasma membranes found in most cell types. The protein links integrin subunits to the tyrosine kinase FYN, an initiating step in coupling integrins to the Ras-ERK pathway and promoting cell cycle progression. The gene is a tumor suppressor gene candidate and a negative regulator of the Ras-p42/44 mitogen-activated kinase cascade. Caveolin 1 and caveolin 2 are located next to each other on chromosome 7 and express colocalizing proteins that form a stable hetero-oligomeric complex. Mutations in this gene have been associated with Berardinelli-Seip congenital lipodystrophy. Alternatively spliced transcripts encode alpha and beta isoforms of caveolin 1. CAV1 may act as a scaffolding protein within caveolar membranes; Interacts directly with G-protein alpha subunits and can functionally regulate their activity (By similarity); Involved in the costimulatory signal essential for T-cell receptor (TCR)-mediated T-cell activation. Its binding to DPP4 induces T-cell proliferation and NF-kappa-B activation in a T-cell receptor/CD3-dependent manner. Recruits CTNNB 1 to caveolar membranes and may regulate CTNNB 1-mediated signaling through the Wnt pathway.

Caveolin-1 is a protein that in humans is encoded by the CAV1 gene. The scaffolding protein encoded by this gene is the main component of the caveolae plasma membranes found in most cell types. The protein links integrin subunits to the tyrosine kinase FYN, an initiating step in coupling integrins to the Ras-ERK pathway and promoting cell cycle progression. The gene is a tumor suppressor gene candidate and a negative regulator of the Ras-p42/44 MAP kinase cascade. CAV1 and CAV2 are located next to each other on chromosome 7 and express colocalizing proteins that form a stable hetero-oligomeric complex. By using alternative initiation codons in the same reading frame, two isoforms (alpha and beta) are encoded by a single transcript from this gene. CAV1 biomarkers include those described by Haeusler et al., Association of a CAV-1 haplotype to familial aggressive prostate cancer. Prostate 2005 65(2):171-7; Li et al., Caveolin-1 maintains activated Akt in prostate cancer cells through scaffolding domain binding site interactions with and inhibition of serine/threonine protein phosphatases PP1 and PP2A. Mol Cell Biol. 2003 23(24):9389-404; De Vizio et al., An absence of stromal caveolin-1 is associated with advanced prostate cancer, metastatic disease and epithelial Akt activation. Cell Cycle 2009 8(15):2420-4; Ayala et al., Stromal antiapoptotic paracrine loop in perineural invasion of prostatic carcinoma. Cancer Res. 2006 66(10):5159-64.

The biomarkers include DES (desmin) gene products. This gene encodes a muscle-specific class HI intermediate filament. Homopolymers of this protein form a stable intracytoplasmic filamentous network connecting myofibrils to each other and to the plasma membrane. Mutations in this gene are associated with desmin-related myopathy, a familial cardiac and skeletal myopathy (CSM), and with distal myopathies. Desmin are class-III intermediate filaments found in muscle cells. In adult striated muscle they form a fibrous network connecting myofibrils to each other and to the plasma membrane from the periphery of the Z-line structures. DES biomarkers include those described by Yeh et al., Malignancy arising in seminal vesicles in the transgenic adenocarcinoma of mouse prostate (TRAMP) model. Prostate 2009 69(7):755-60; Jun et al., Primary synovial sarcoma of the prostate: report of 2 cases and literature review. Int J Surg Pathol. 2008 16(3):329-34; Tewari et al., Identification of the retrotrigonal layer as a key anatomical landmark during robotically assisted radical prostatectomy. BJU Int. 2006 98(4):829-32; Ayala et al., Reactive stroma as a predictor of biochemical-free recurrence in prostate cancer. Clin Cancer Res. 2003 9(13):4792-801.

The biomarkers include DPYSL3 (dihydropyrimidinase-like 3) gene products: DPYSL3 is necessary for signaling by class 3 semaphorins and subsequent remodeling of the cytoskeleton; Plays a role in axon guidance, neuronal growth cone collapse and cell migration (By similarity).

DNA microarray analysis of TGFBR3 knockdown in RWPE-1 cells showed that expression of DPYSL3, Vimentin, COCH, SERPINFL PMP22, LTBP1 and BMP4 were decreased both with TGFBR3 knockdown and in the transition to prostate cancer. DPYSL3 gene products include those described by Thomson Okatsu et al., Method for the Molecular Diagnosis of Prostate Cancer and Kit for Implementing Same. United States Patent Application 20100227317; Sharifi et al., TGFBR3 loss and consequences in prostate cancer. Prostate 2007 67(3):301-11; Sharifi et al., TGFBR3 is lost in prostate cancer, contributing to malignant transformation. J Clin. Onc. ASCO Annual Meeting Proceedings (Post-Meeting Edition) 2006 24(18S):14552.

The biomarkers include FHL1 (four and a half LIM domains 1) gene products: This gene encodes a member of the four-and-a-half-LIM-only protein family. Family members contain two highly conserved, tandemly arranged, zinc finger domains with four highly conserved cysteines binding a zinc atom in each zinc finger. Expression of these family members occurs in a cell- and tissue-specific mode and these proteins are involved in many cellular processes. Mutations in this gene have been found in patients with Emery-Dreifuss muscular dystrophy. Multiple alternately spliced transcript variants which encode different protein isoforms have been described. FHL1 may have an involvement in muscle development or hypertrophy.

Four and a half LIM domains protein 1 is a protein that in humans is encoded by the FHL1 gene. LIM proteins, named for ‘LIN11, ISL1, and MEC3,’ are defined by the possession of a highly conserved double zinc finger motif called the LIM domain. FHL1 expression is suppressed in tumors of the breast, kidney, and prostate. The FHL1 biomarkers include those described by Li et al., Coordinate suppression of Sdpr and FhL1 expression in tumors of the breast, kidney, and prostate. Cancer Sci. 2008 99(7):1326-33; Lee et al., Chromosomal mapping, tissue distribution and cDNA sequence of four-and-a-half LIM domain protein 1 (FHL1). Gene 1998 216(1):163-70.

The biomarkers include SVIL (supervillin) gene products (SVIL; Gene ID: 6840; AKA p205/p250; DKFZp686A17191; archvillin; membrane-associated F-actin binding protein, p205 Archvillin). This gene encodes a bipartite protein with distinct amino- and carboxy-terminal domains. The amino-terminus contains nuclear localization signals and the carboxy-terminus contains numerous consecutive sequences with extensive similarity to proteins in the gelsolin family of actin-binding proteins, which cap, nucleate, and/or sever actin filaments. The gene product is tightly associated with both actin filaments and plasma membranes, suggesting a role as a high-affinity link between the actin cytoskeleton and the membrane. The encoded protein appears to aid in both myosin II assembly during cell spreading and disassembly of focal adhesions. Two transcript variants encoding different isoforms of supervillin have been described. SVIL forms a high-affinity link between the actin cytoskeleton and the membrane. Isoform 1 (archvillin) is among the first costameric proteins to assemble during myogenesis and it contributes to myogenic membrane structure and differentiation; Appears to be involved in myosin II assembly; May modulate myosin II regulation through MLCK during cell spreading, an initial step in cell migration; May play a role in invadopodial function. Isoform 2 may be involved in modulation of focal adhesions. Supervillin-mediated down-regulation of focal adehesions involves binding to TRIP6 (By similarity).

Of significance to prostate cancer biology is SVIL's possible involvement in the regulation of androgen receptor known as a major regulator of prostate cancer. A number of treatment modalities involve androgen receptor inhibitors and resistance to such inhibitors is accompanied by cancer progression and metastasis. SVIL has been shown to interact with androgen receptor.

Significant down-regulation in the mRNA expression levels of PRIMA1, TU3A, PDLIM4, FLJ14084, SVIL, SORBS1, C21orf63, and KIAA1210 and up-regulation of FABP5, SOX4, and MLP in prostate cancer tissues has been discussed in the literature. SVIL biomarkers include those described by Ting et al., Supervillin associates with androgen receptor and modulates its transcriptional activity. Proc Natl Acad Sci USA 2002 99(2):661-6; Sampson et al., Identification and characterization of androgen receptor associated coregulators in prostate cancer cells. J Biol Regul Homeost Agents 2001 15(2):123-9; Tasseff et al., Analysis of the molecular networks in androgen dependent and independent prostate cancer revealed fragile and robust subsystems. PLoS One 2010 5(1):e8864; Vanaja et al., PDLIM4 repression by hypermethylation as a potential biomarker for prostate cancer. Clin Cancer Res. 2006 12(4):1128-36.

SVIL was discovered to show significant expression changes on the RNA level and has been shown in prostate tissue staining experiments to be readily detectable in prostate stroma and in tumor epithelial tissue structures. Evidence of differential expression of SVIL in formalin fixed tissue sections labeled with anti-SVIL antibodies indicates that this marker has value as a diagnostic marker as well as a control marker, either alone or in conjunction with other biomarkers.

The biomarkers include VIM (vimentin) gene products. This gene encodes a member of the intermediate filament family. Intermediate filamentents, along with microtubules and actin microfilaments, make up the cytoskeleton. The protein encoded by this gene is responsible for maintaining cell shape, integrity of the cytoplasm, and stabilizing cytoskeletal interactions. It is also involved in the immune response, and controls the transport of low-density lipoprotein (LDL)-derived cholesterol from a lysosome to the site of esterification. It functions as an organizer of a number of critical proteins involved in attachment, migration, and cell signaling. Mutations in this gene causes a dominant, pulverulent cataract. Vimentins are class-III intermediate filaments found in various non-epithelial cells, especially mesenchymal cells.

Vimentin is a member of the intermediate filament family of proteins that is especially found in connective tissue. Intermediate filaments are an important structural feature of eukaryotic cells. They, along with microtubules and actin microfilaments, make up the cytoskeleton. Although most intermediate filaments are stable structures, in fibroblasts, vimentin exists as a dynamic structure. This filament is used as a marker for mesodermally derived tissues, and as such can be used as an immunohistochemical marker for sarcomas. It has been used as a sarcoma tumor marker to identify mesenchyme and discussed as a promising marker for predicting the invasion and metastasis of prostate cancer cells.

There was high risk of biochemical recurrence associated with tumors that displayed high levels of expression of TGF-beta1, vimentin, and NF-kappaB and low level of cytokeratin 18. This was particularly true for vimentin, which is independent of patients' Gleason score. It has been suggest that vimentin affects prostate cancer cells motility and invasiveness. The VIM biomarkers include those described by Leader et al., Vimentin: an evaluation of its role as a tumour marker. Histopathology 1987 11(1):63-72; Wei et al., Effects of vimentin on invasion and metastasis of prostate cancer cell lines PC-3M-1E8 and PC-3M-2B4. Ai Zheng 2008 27(1):30-4; Zhang et al., Nuclear factor-kappaB-mediated transforming growth factor-beta-induced expression of vimentin is an independent predictor of biochemical recurrence after radical prostatectomy. Clin Cancer Res. 2009 15(10):3557-67; Zhao et al., Vimentin affects the mobility and invasiveness of prostate cancer cells. Cell Biochem Funct. 2008 26(5):571-7.

Diagnostic Stroma Biomarkers

The biomarkers include ALDH3A2 (aldehyde dehydrogenase 3 family, member A2) gene products. Aldehyde dehydrogenase isozymes are thought to play a major role in the detoxification of aldehydes generated by alcohol metabolism and lipid peroxidation. This gene product catalyzes the oxidation of long-chain aliphatic aldehydes to fatty acid. Mutations in the gene cause Sjogren-Larsson syndrome. Alternatively spliced transcript variants encoding different isoforms have been found for this gene. ALDH3A2 catalyzes the oxidation of long-chain aliphatic aldehydes to fatty acids; Active on a variety of saturated and unsaturated aliphatic aldehydes between 6 and 24 carbons in length. THE ALDH3A2 biomarkers include those described by van den Hoogen et al., High aldehyde dehydrogenase activity identifies tumor-initiating and metastasis-initiating cells in human prostate cancer. Cancer Res. 2010 70(12):5163-73.

The biomarkers include PDLIM7 (PDZ and LIM domain 7 (enigma)) gene products. The protein encoded by this gene is representative of a family of proteins composed of conserved PDZ and LIM domains. LIM domains are proposed to function in protein-protein recognition in a variety of contexts including gene transcription and development and in cytoskeletal interaction. The LIM domains of this protein bind to protein kinases, whereas the PDZ domain binds to actin filaments. The gene product is involved in the assembly of an actin filament-associated complex essential for transmission of ret/ptc2 mitogenic signaling. The biological function is likely to be that of an adapter, with the PDZ domain localizing the LIM-binding proteins to actin filaments of both skeletal muscle and nonmuscle tissues. Alternative splicing of this gene results in multiple transcript variants. PDLIM7 may function as a scaffold on which the coordinated assembly of proteins can occur; May play a role as an adapter that, via its PDZ domain, localizes LIM-binding proteins to actin filaments of both skeletal muscle and nonmuscle tissues; Involved in both of the two fundamental mechanisms of bone formation, direct bone formation e.g (embryonic flat bones mandible and cranium), and endochondral bone formation e.g (embryonic long bone development); Plays a role during fracture repair; Involved in BMP6 signaling pathway (By similarity).

PDZ and LIM domain protein 7 is a protein that in humans is encoded by the PDLIM7 gene. The protein encoded by this gene is representative of a family of proteins composed of conserved PDZ and LIM domains. LIM domains are proposed to function in protein-protein recognition in a variety of contexts including gene transcription and development and in cytoskeletal interaction. The LIM domains of this protein bind to protein kinases, whereas the PDZ domain binds to actin filaments. The gene product is involved in the assembly of an actin filament-associated complex essential for transmission of ret/ptc2 mitogenic signaling. The biological function is likely to be that of an adapter, with the PDZ domain localizing the LIM-binding proteins to actin filaments of both skeletal muscle and nonmuscle tissues. Alternative splicing of this gene results in multiple transcript variants. PDLIM7 has been shown to interact with TPM2 (Guy et al. Mol. Biol. Cell 1999). The PDLIM7 biomarkers include those described by Guy et al., The PDZ domain of the LIM protein enigma binds to beta-tropomyosin. Mol Biol Cell 1999 10(6):1973-84; Talantov et al., Gene based prediction of clinically localized prostate cancer progression after radical prostatectomy. J Urol. 2010 184(4):1521-8; Davila et al., LIM kinase 1 is essential for the invasive growth of prostate epithelial cells: implications in prostate cancer. J Biol Chem. 2003 278(38):36868-75; Krcmery et al., Nucleo-cytoplasmic functions of the PDZ-LIM protein family: new insights in organ development. Bioessays 2010 32(2):100-8.

The biomarkers include COL4A2 (collagen, type IV, alpha 2) gene products: This gene encodes one of the six subunits of type IV collagen, the major structural component of basement membranes. The C-terminal portion of the protein, known as canstatin, is an inhibitor of angiogenesis and tumor growth. Like the other members of the type IV collagen gene family, this gene is organized in a head-to-head conformation with another type IV collagen gene so that each gene pair shares a common promoter. Type IV collagen is the major structural component of glomerular basement membranes (GBM), forming a ‘chicken-wire’ meshwork together with laminins, proteoglycans and entactin/nidogen. Canstatin, a cleavage product corresponding to the collagen alpha 2(IV) NC1 domain, possesses both anti-angiogenic and anti-tumor cell activity. It inhibits proliferation and migration of endothelial cells, reduces mitochondrial membrane potential, and induces apoptosis. Specifically induces Fas-dependent apoptosis and activates procaspase-8 and -9 activity. Ligand for alphavbeta3 and alphavbeta5 integrins.

Collagen alpha-2(IV) chain is a protein that in humans is encoded by the COL4A2 gene. This gene encodes one of the six subunits of type IV collagen, the major structural component of basement membranes. The C-terminal portion of the protein, known as canstatin, is an inhibitor of angiogenesis and tumor growth. Like the other members of the type IV collagen gene family, this gene is organized in a head-to-head conformation with another type IV collagen gene so that each gene pair shares a common promoter. COL4A2 biomarkers include those described by He et al., The C-terminal domain of canstatin suppresses in vivo tumor growth associated with proliferation of endothelial cells. Biochem Biophys Res Commun. 2004 318(2):354-60; Xu et al., Inherited genetic variant predisposes to aggressive but not indolent prostate cancer. Proc Natl Acad Sci USA 2010 107(5):2136-40; Saleem et al., S100A4 accelerates tumorigenesis and invasion of human prostate cancer through the transcriptional regulation of matrix metalloproteinase 9. Proc Natl Acad Sci USA 2006 103(40):14825-30; Kamphaus et al., Canstatin, a novel matrix-derived inhibitor of angiogenesis and tumor growth. J Biol Chem. 2000 275(2):1209-15.

The biomarkers include HSPB8 (heat shock 22 kDa protein 8) gene products: A protein encoded by this gene belongs to the superfamily of small heat-shock proteins containing a conservative alpha-crystallin domain at the C-terminal part of the molecule. The expression of this gene in induced by estrogen in estrogen receptor-positive breast cancer cells, and this protein also functions as a chaperone in association with Bag3, a stimulator of macroautophagy. Thus, this gene appears to be involved in regulation of cell proliferation, apoptosis, and carcinogenesis, and mutations in this gene have been associated with different neuromuscular diseases, including Charcot-Marie-Tooth disease. HSPB8 displays temperature-dependent chaperone activity.

HSPB8 has been shown to interact with Hsp27 and HSPB2. Among these were several known prostate cancer relevant genes, such as AMACR, TARP, LIM, GPR160 (all up-regulated), CAV1, NTN1, MT1X; CLU, TRIM29, SPARCL1 and HSPB8 (all down-regulated) (Schlomm et al. Int. J Oncol. 2005). HSPB8 biomarkers include those described by Sun et al., Interaction of human HSP22 (HSPB8) with other small heat shock proteins. J Biol Chem. 2004 279(4):2394-402; Reily et al., Rapid imaging of human melanoma xenografts using an scFv fragment of the human monoclonal antibody H11 labelled with 111In. Nucl Med Commun. 2001 22(5):587-95; Schlomm et al., Extraction and processing of high quality RNA from impalpable and macroscopically invisible prostate cancer for microarray gene expression analysis. Int J Oncol. 2005 27(3):713-20; Berretta et al., Cancer biomarker discovery: the entropic hallmark. PLoS One 2010 5(8):e12262.

The biomarkers include FBN1 (fibrillin 1) gene products: This gene encodes a member of the fibrillin family. The encoded protein is a large, extracellular matrix glycoprotein that serve as a structural component of 10-12 nm calcium-binding microfibrils. These microfibrils provide force bearing structural support in elastic and nonelastic connective tissue throughout the body. Mutations in this gene are associated with Marfan syndrome, isolated ectopia lentis, autosomal dominant Weill-Marchesani syndrome, MASS syndrome, and Shprintzen-Goldberg craniosynostosis syndrome. Fibrillins are structural components of 10-12 nm extracellular calcium-binding microfibrils, which occur either in association with elastin or in elastin-free bundles. Fibrillin-1-containing microfibrils provide long-term force bearing structural support.

Fibrillin-1 is a protein that in humans is encoded by the FBN1 gene. This gene encodes a member of the fibrillin family. The encoded protein is a large, extracellular matrix glycoprotein that serve as a structural component of 10-12 nm calcium-binding microfibrils. These microfibrils provide force bearing structural support in elastic and nonelastic connective tissue throughout the body. Mutations in this gene are associated with Marfan syndrome, isolated ectopia lentis, autosomal dominant Weill-Marchesani syndrome, MASS syndrome, and Shprintzen-Goldberg craniosynostosis syndrome. The FBN1 biomarkers include those described by Murabito et al., A genome-wide association study of breast and prostate cancer in the NHLBI's Framingham Heart Study. BMC Med Genet. 2007 8 Suppl 1:S6; Wang et al., Survey of differentially methylated promoters in prostate cancer cell lines. Neoplasia 2005 7(8):748-60; Prodoehl et al., Fibrillins and latent TGFbeta binding proteins in bovine ovaries of offspring following high or low protein diets during pregnancy of dams. Mol Cell Endocrinol. 2009 307(1-2):133-41; Booms et al., Differential effect of FBN1 mutations on in vitro proteolysis of recombinant fibrillin-1 fragments. Hum Genet. 2000 107(3):216-24.

The provided biomarkers further include biomarkers described in Examples 1-3.

Various aspects of the invention are further described and illustrated by way of the several examples which follow, none of which are intended to limit the scope of the invention.

### Example 1

### Identification of Stroma Biomarkers

This Example describes the identification of gene expression changes in prostate stroma and identification of stroma biomarkers useful in detection of nearby lesions, e.g., prostate tumors. A stroma-specific classifier for nearby tumor was constructed based on 114 stroma biomarker genes.

A. Materials and Methods

Biological Samples

Biological samples used in the study were obtained from biopsies from prostate cancer patients, normal donors, and rapid autopsy biopsy. Table 1, below, lists the sources of biological samples for datasets (1-4) used in this study.

Dataset 1 contained expression data from multiple sources. For example, Dataset 1 included data from 109 post-prostatectomy frozen tissue samples from 87 patients. These samples were post-prostatectomy frozen tissue samples obtained by informed consent using IRB-approved and HIPPA-compliant protocols. All tissues, except where noted, were collected at surgery and escorted to pathology for expedited review, dissection, and snap freezing in liquid nitrogen. Two different types of tissue samples were analyzed from 22 of these 87 patients; one sample type was enriched for tumor. The other sample type contained stroma from cases of prostate cancer, but with this stroma generally located more than 15 mm from the tumor, and usually in the contralateral lobe. Dataset 1 further included expression data from 27 prostate biopsy specimens obtained as fresh snap frozen biopsy cores from 18 normal prostates. These samples were obtained from the control untreated subjects of a clinical trial to evaluate the role of Difluoromethylornithine (DFMO) to decrease the prostate size of normal men. Ten of these were collected before the treatment period, and eight were collected after the treatment period had ended (Simoneau A R, Gerner E W, Nagle R, et al. “The effect of difluoromethylornithine on decreasing prostate size and polyamines in men: results of a year-long phase IIb randomized placebo-controlled chemoprevention trial,” Cancer Epidemiol Biomarkers Prev 2008; 17(2):292-9). Finally, Dataset 1 included expression data from 13 prostates from non-prostate cancer donors, obtained from the rapid autopsy program of the Sun Health Research Institute, with an average patient age of 82 years, frozen within 6 hours of demise.

Dataset 2 includes expression data from 136 samples from 82 prostate cancer patients. These samples were post-prostatectomy frozen tissue samples obtained by informed consent using IRB-approved and HIPPA-compliant protocols. All tissues, except where noted, were collected at surgery and escorted to pathology for expedited review, dissection, and snap freezing in liquid nitrogen. Expression data from 65 samples including predominately tumor was used as a test dataset. 71 of the tumor-bearing samples were manually microdissected to obtain tumor-adjacent stroma which was used for validation of the Diagnostic Classifier, described below.

Datasets 3 and 4, used as test sets, were independently developed (Table 1). Dataset 3 included a series of 79 samples (Stephenson A J, Smith A, Kattan M W, et al. “Integration of gene expression profiling and clinical variables to predict prostate carcinoma recurrence after radical prostatectomy,” Cancer 2005; 104(2):290-8; Sun Y, Goodison S., “Optimizing molecular signatures for predicting prostate cancer recurrence,” Prostate 2009; 69(10):1119-27). Dataset 4 (Liu P, Ramachandran S, Ali Seyed M, et al., “Sex-determining region Y box 4 is a transforming oncogene in human prostate cancer cells,” Cancer Res 2006; 66(8):4011-9 [data available at http://www.ebi.ac.uk/arrayexpress/browse.html?keywords=E-TABM-26]) included 57 samples from 44 patients, including 13 samples of stroma near tumor and 44 tumor-bearing samples.

TABLE 1
Datasets used in analysis of biomarker expression

| | | Subject | Array | Array: Tumor/ | |
|---|---|---|---|---|---|
| Dataset | Platform | Number | Number | Nontumor/Normal | Reference |
| 1 | U133Plus2 | P = 87 | 108 | 68/40/0 | GSE17951 |
| Training + | | B = 18 | 27 | 0/0/27 | |
| test | | A = 13 | 13 | 0/0/13 | |
| 2 | U133A | P = 82 | 136 | 65/71/0 | GSE08218 |
| 3 | U133A | P = 79 | 79 | 79/0/0 | Provided byWilliam L. Gerald |
| | | | | | (Stephenson AJ, Smith A, |
| | | | | | Kattan MW, et al., “Integration |
| | | | | | of gene expression profiling |
| | | | | | and clinical variables to predict |
| | | | | | prostate carcinoma recurrence |
| | | | | | after radical prostatectomy,” |
| | | | | | Cancer |
| | | | | | 104:290-8, 2005) |
| 4 | U133A | P = 44 | 57 | 44/13/0 | http://www.ebi.ac.uk/ |
| | | | | | microarrayas/ae/ |
| | | | | | browse.html?keywords=ET |
| | | | | | ABM- |
| | | | | | 26 |

P = Samples from prostate cancer patients;
B = Biopsies from normal donors.
A = Prostate donated by rapid autopsy.
Datasets 1 and 2 were collected from five participating institutions in San Diego County, CA. Demographic, pathology; clinical values were individually recorded in shadow charts and maintained in the UCI SPECS consortium database

Preparation of RNA and Expression Analysis

RNA for expression analysis was prepared directly from frozen tissue following dissection of OCT (optimum cutting temperature compound) blocks with the aid of a cryostat. For expression analysis, 50 micrograms (10 micrograms for biopsy tissue) of total RNA samples were processed for hybridization to Affymetrix® GeneChips®. As indicated in Table 1, expression analysis for all samples for Dataset 1 was assessed using the U133 Plus 2.0 platform; the U133A platform was used for Dataset 2. Data for Datasets 1 and 2 were deposited in the Gene Expression Omnibus (GEO) database (http://www.ncbi.nlm.nih.gov/geo), referenced by accession numbers GSE17951 (Dataset 1) and GSE8218 (Dataset 2). For Datasets 1 and 2, the distributions for the four principal cell types (tumor epithelial cells, stroma cells, epithelial cells of BPH, and epithelial cells of dilated cystic glands) were estimated by three (Dataset 1) and four (Dataset 2) pathologists, whose estimates were averaged as described by Stuart R O, Wachsman William, Berry Charles C., Arden Karen, Goodison Steven, Klacansky Igor, McClelland Michael, Wang-Rodriquez Jessica, Wasserman Linda, Sawyers, Ann, Yipeng, Wang, Kalcheva, Iveata, Tarin David, Mercola Dan, “In silico dissection of celltype associated patterns of gene expression in prostate cancer,” Proceeding of the National Academy of Sciences USA 2004; 101:615-20. Expression analysis of Datasets 3 and 4 was carried out using the U133A platform.

Manual Microdissection

71 of the tumor-bearing samples of Dataset 2 were manually microdissected to obtain tumor-adjacent stroma which was used for validation of the Diagnostic Classifier. For manual microdissection, the tumor-bearing tissue was embedded in an OCT (optimum cutting temperature compound, Fisher Scientific Inc.) block, then mounted in a cryostat. Frozen sections were stained using hematoxylin and eosin (H and E) to visualize the location of the tumor. A border between tumor and adjacent stroma was marked on the glass slide using a Pilot Ultrafine Point Pen which was used as a guide to locate the border on the OCT-block surface. Then the OCT-embedded block was etched with a single straight cut with a scalpel (˜1 mm deep) to divide the embedded tissue into a tumor zone and tumor-adjacent stroma. Subsequent cryosections produced two halves at the site of the etched cut and were separately used for H&E staining and examined to confirm their composition. Multiple subsequent frozen sections of the tumor-adjacent stroma half were then pooled and used for RNA preparation and microarray hybridization. A final frozen section was used for H&E staining and examined to confirm that the tumor-adjacent stroma remained free of tumor cells. The pooled tumor-adjacent stroma was then used for RNA preparation and expression analysis.

Statistical Tools Implemented in R

The U133 Plus 2.0 platform used for Dataset 1 had about 55,000 probe sets; the U133A used for Datasets 2, 3 and 4, contained 22,000 probe sets. Normalization was carried out across multiple datasets using the ˜22,000 probe sets in common to all Datasets. First, Dataset 1 was quantile-normalized using the function ‘normalizeQuantiles’ of LIMMA routine (Dalgaard P., “Statistics and Computing: Introductory Statistics with R,” pp. 260, Springer-Verlag Inc., NY. 2002). Datasets 2-4 were then quantile-normalized by referencing normalized Dataset 1 using a modified function ‘REFnormalizeQuantiles’ which was coded by ZJ and is available at the SPECS website (available at http://www.pathology.uci.edu/faculty/mercola/UCISPECSHorne.html).

The LIMMA package from Bioconductor was used to detect differentially expressed genes. Prediction Analysis of Microarray (PAM (Guo Y, Hastie T, Tibshirani R, “Regularized linear discriminant analysis and its application in microarrays,” Biostatistics 2007; 8(1):86-100)), implemented in R, was used to develop an expression-based classifier from the training sets and then applied to the test sets without further change.

A multiple linear regression (MLR) model was used to fit gene expression data, and known percent cell-type composition for four cell types to estimate expression coefficients for each cell component, to describe the observed Affymetrix intensity of a gene as the summation of the contributions from different types of cells given the pathological cell constitution data:

$$g = {β}_{0} + {\sum }_{j = 1}^{C} {β}_{j} {p}_{j} + e , \qquad Equation ( 1 )$$

where g is the expression value for a gene, p's are the percentage data determined by the pathologists, and β's are the expression coefficients associated with different cell types. In model (1), C is the number of tissue types under consideration.

Three major tissue types were included, i.e., tumor, stroma and BPH (Benign Prostate Hyperplasia). βⱼ was the estimate of the relative expression level in cell type j (i.e. the expression coefficient) compared to the overall mean expression level. The regression model was applied to the patient cases in Dataset 1 to obtain the model parameters (β's) and their corresponding p-values, which were then used to aid subsequent gene screening.

The application to prostate cancer expression data and validation by immunohistochemistry and by correlation of derive βⱼ values with LCM-derived samples assayed by qPCR has been described (Stuart R O, Wachsman William, Berry Charles C., Arden Karen, Goodison Steven, Klacansky Igor, McClelland Michael, Wang-Rodriquez Jessica, Wasserman Linda, Sawyers, Ann, Yipeng, Wang, Kalcheva, Iveata, Tarin David, Mercola Dan. In silico dissection of celltype associated patterns of gene expression in prostate cancer. Proceeding of the National Academy of Sciences USA 2004; 101:615-20).

The cell-type specific expression coefficients (β's) were used to identify genes largely expressed in stroma using three criteria: (1) Genes that are expressed in tumor epithelial cells at greater than 10% of the expression in stroma cells, i.e., βₛ>10×β_{T}, where βₛ and β_{T} are defined as for Equation 1 above. (2) βₛ>0; and (3) p(βₛ)<0.1. Criteria (2) and (3) selected genes that are significantly expressed in stroma cells. In the MLR model, criterion (3) had two implications: either the gene is expressed in stroma cells but not in tumor cells (βₛ>0 and β_{T}<0) and is retained or the gene is expressed in both stroma cells and tumor cells (βₛ>0 and β_{T}>0) but is only retained if (βₛ>10×β_{T}).

For Datasets 1 and 2, the distributions for the four principal cell types (tumor epithelial cells, stroma cells, epithelial cells of BPH, and epithelial cells of dilated cystic glands) were estimated. A frozen section was taken immediately above the sections pooled for RNA preparation and again immediately below the pooled sections. Each of these extra sections was reviewed by three (Dataset 1) or four (Dataset 2) pathologists whose estimates were averaged as described (Stuart R O, Wachsman William, Berry Charles C., Arden Karen, Goodison Steven, Klacansky Igor, McClelland Michael, Wang-Rodriquez Jessica, Wasserman Linda, Sawyers, Ann, Yipeng, Wang, Kalcheva, Iveata, Tarn David, Mercola Dan. In silico dissection of celltype associated patterns of gene expression in prostate cancer. Proceeding of the National Academy of Sciences USA 2004; 101:615-20). The estimates exhibited an overall agreement of 4.3% standard deviation for the four estimated cell types. The resulting significantly differentially expressed genes for the comparison of normal prostate biopsies to tumor-bearing prostate tissue were used for development of the diagnostic classifier.

B. Identification of Stroma-Derived Genes

Expression profiles of 15 normal biopsy samples and 13 tumor-adjacent stroma samples from prostatectomies were compared using a permutation strategy to enhance detection of significant differences. A three-step process was used to confirm that stroma within and directly adjacent to prostate cancer epithelial cells exhibits significant RNA expression changes compared to normal prostate stroma. In step (1), genes were identified that were differentially expressed in tumor-adjacent stroma compared to normal stroma. In step (2), a stroma-specific set of differentially expressed genes was created by filtering these differences (by removing age-related genes and those genes also expressed in tumor cells). Step 3 was performed in light of the limiting number of normal biopsies; steps (1) and (2) were repeated using a permutation procedure, which greatly enhanced the extraction of information in the normal biopsies.

(1) Identification of Genes Differentially Expressed in Tumor-Adjacent and Normal Stroma

In step (1) Affymetrix gene expression data were acquired from normal frozen biopsies from each of 15 subjects that were judged to be free of cancer by histological examination of the six cores of the volunteer biopsies (Simoneau A R, Gerner E W, Nagle R, et al., “The effect of difluoromethylornithine on decreasing prostate size and polyamines in men: results of a year-long phase IIb randomized placebo-controlled chemoprevention trial,” Cancer Epidemiol Biomarkers Prev 2008; 17(2):292-9).

Data from 13 of these 15 samples (with two samples held in reserve for permutation analysis in step (3)) were compared to the gene expression data for 13 tumor-bearing patient cases from Dataset 1 selected with tumor cell content (T) greater than 0% but less than 10% tumor cell content (average stroma content ˜80%). These criteria ensured that the majority of stroma tissues included from the cancer-positive patients was close to tumor, while T<10% ensured that the impact from tumor cells is minimal to allow capture of altered expression signals from stroma cells rather than tumor cells. Using a moderated t-test implemented in the LEMMA package of R (25), this comparison yielded 3888 significant expression changes between these two groups with a p value <0.05. We used a relatively relaxed p value cutoff for the first-step of feature selection to allow more genes to enter subsequent screening steps. The 3888 probe sets were composed of a nearly equal number of up- and down-regulated genes. There was a substantial difference in age between the normal stroma group (average age=51.9 years) and the near-tumor stroma group (average age=60.6 years).

(2) Filtering Differences to Identify Stroma Biomarkers

In step (2), the roughly 3800 significant gene expression changes from step (1) were filtered to exclude genes known to be expressed at similar levels in epithelial tumor cells and to remove genes that change with age. Overall gene expression of the 13 normal stroma samples used for training was compared with 13 normal prostate specimens obtained by rapid autopsy as described in Example 1A (Materials and Methods), with an average age of 82. Prostate glands from the rapid autopsy series with an average age of 84 years exhibited a markedly increased heterogeneity of gland shapes with stroma containing increased fibroblast and myofibroblast-like cells. The comparison revealed 8898 significant expression changes (p value <0.05). 1678 of these probe sets were also detected in the comparison of normal stroma samples to stroma near tumor. After eliminating all of these potential aging-related genes, the remaining 2210 probe sets included nearly equal numbers of up- and down-regulated genes.

Some differential expression in this comparison may have represented expression changes specific to the residual tumor cells or epithelium cells in some samples, rather than changes between two types of stromal cells. To reduce the possibility that epithelial cell derived expression changes might influence subsequent results, genes that appeared to be expressed in tumor at 10% or more of the expression in stroma were removed. Because even “pure” tumor samples can be contaminated with stroma, risking the elimination of genes expressed only in stroma, identification of genes expressed in tumor was achieved using multiple linear regression (MLR) analysis as described in Example 1A (Materials and Methods), above.

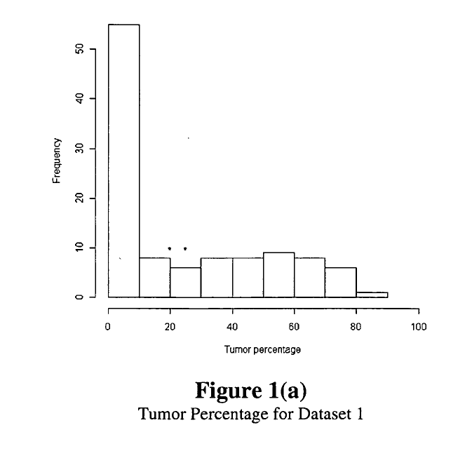

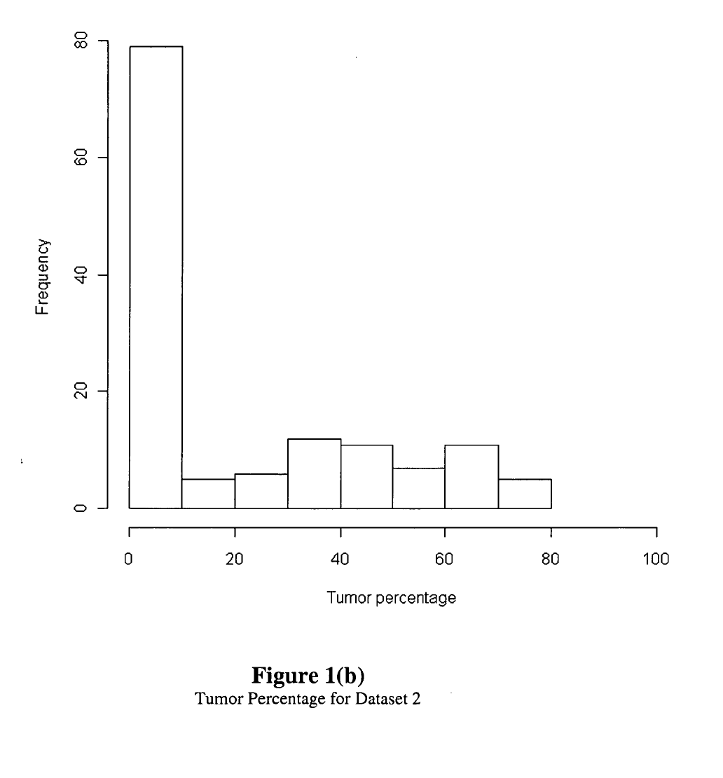

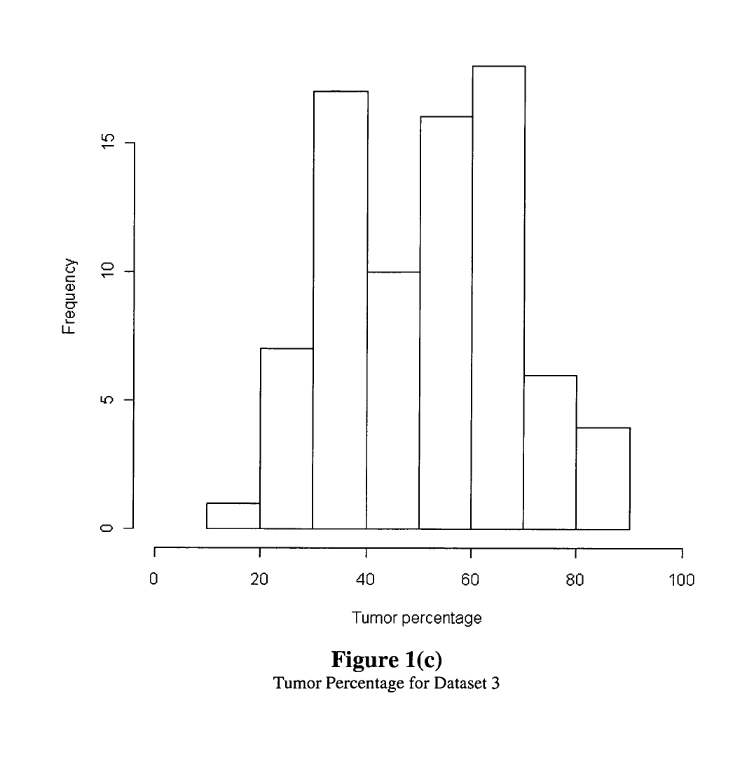

The percent cell composition of 108 samples from 87 patients in Dataset 1, intentionally encompassing a wide range of tissue percentages, was determined by a panel of three pathologists as described in Example 1A (Materials and Methods). The distribution is shown in FIG. 1(a). Model diagnostics showed that the fitted model for genes significantly expressed in tumor or stroma accounted for >70% of the total variation (i.e., the variation of error, e in Equation 1, was <30% of the total variation), indicating a plausible modeling scheme.

Of 2210 probe sets, derived above, 160 probe sets were obtained that were predominantly expressed in stroma cells and exhibited differential expression between near-tumor stroma and normal stroma. The average expression of these 160 probe sets was estimated to be more than twofold greater than the average of all genes expressed in stroma, which was a consequence of the filtering steps for robustness and favored good sensitivity.

(3) Permutation Analysis

Finally in step (3), a permutation analysis was performed. The procedure in step (1) for identifying genes differentially expressed in 13 of the 15 normal stroma biopsies compared to the 13 biopsies of stroma near tumor was repeated using a different selection of 13 biopsy samples from the 15, until all 105 possible combinations of 13 normal biopsy samples drawn from 15 was complete. Filtering for genes associated with aging was carried out as in step (2).

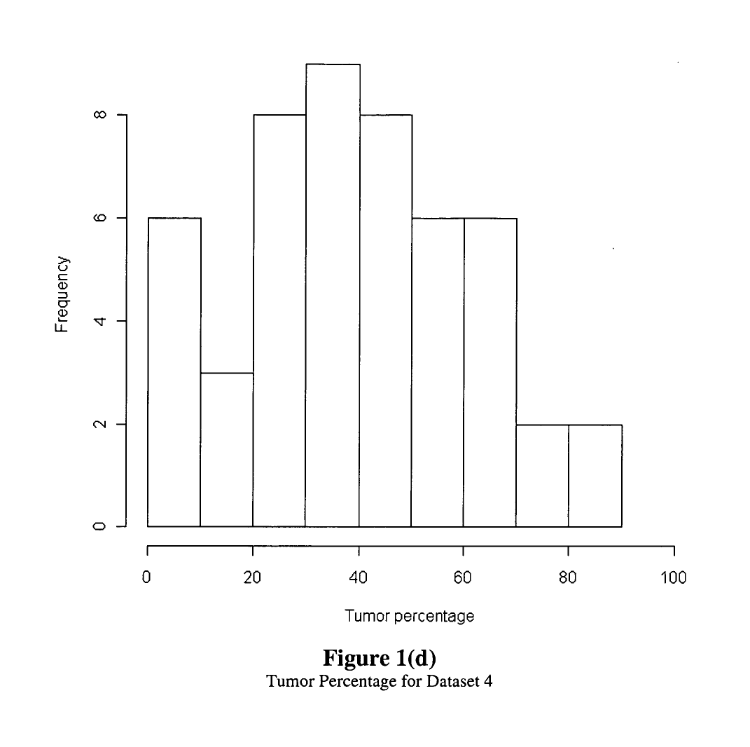

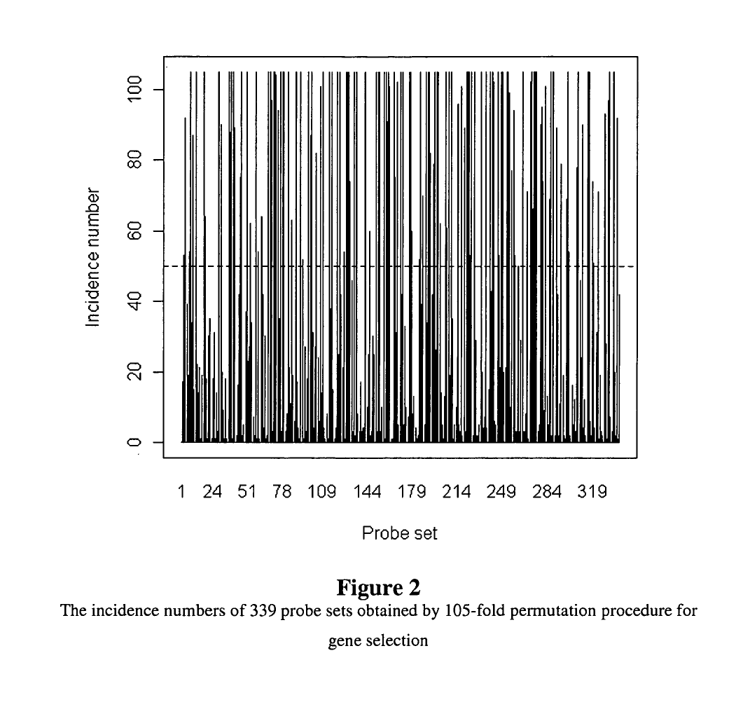

A total of 339 probe sets differentially expressed in stroma near tumor compared to normal stroma were generated by the 105-fold gene selection procedure. Frequency of selection is summarized in FIG. 2. Permutation increased the basis set by 339/160 or over 2-fold. 146 probe sets (listed in Table 3, below) with at least 50 occurrences in the 105-fold permutation were selected for classifier construction.

C. Development of a Diagnostic Classifier

The top ranked 146 probe sets remaining after applying these filters were used for the ten-fold cross-validation procedure of Prediction Analysis for Microarrays (PAM ((described in Tibshirani R, Hastie T, Narasimhan B, Chu G, “Diagnosis of multiple cancer types by shrunken centroids of gene expression.” Proc Natl Acad Sci USA 2002; 99(10):6567-72)) using the same 28 samples used for the initial training. The PAM procedure was used to build a diagnostic classifier.

As shown in Table 2, line 1, the training set included all 15 normal biopsies and the initial 13 samples of stroma near tumor. Of the 146 PAM-input probe sets (see Table 3), 131 probe sets (corresponding to 114 genes) were retained following the 10-fold cross validation procedure of PAM, leading to a prediction accuracy of 96% (Table 2). FIG. 3 presents a “heatmap” of the relative expression of the 131 probe sets among all training samples.

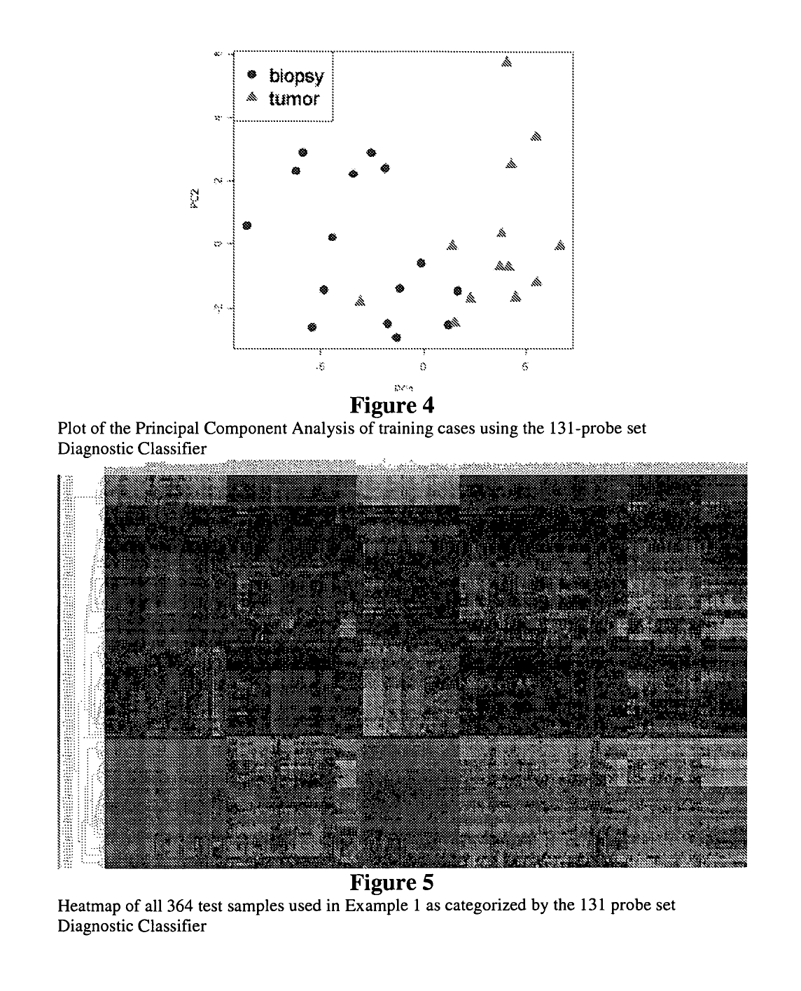

The separation of normal and near-tumor stroma samples of the training set by the classifier is illustrated by the two distinct populations shown in FIG. 4. Thus, the PAM procedure led to a 131 probe set classifier with a training accuracy of 96%.

TABLE 2
Operating Characteristics (OC) for training and testing

| | | | Sample | Accuracy |
|---|---|---|---|---|
| | | Dataset | Number | (%) |
| 1 | Training set | 1 | 28 (15 + 13) | 96.4 |

Test set
Tumor

| | | | | |
|---|---|---|---|---|
| 2 | Tumor-bearing | 1 | 55* | 96.4 |
| 3 | Tumor-bearing | 2 | 65 | 100 |
| 4 | Tumor-bearing | 3 | 79 | 100 |
| 5 | Tumor-bearing | 4 | 44 | 100 |

Normal

| | | | | |
|---|---|---|---|---|
| 6 | Biopsies (1) | 1 | 7 | 100 |
| 7 | Biopsies (2) | 1 | 5 | 60 |
| 8 | Rapid autopsies | 1 | 13 | 92.3 |

Microdissected

| | | | | |
|---|---|---|---|---|
| 9 | Stroma adjacent to tumor | 2 | 71 | 97.1 |
| 10 | Stroma adjacent to tumor | 4 | 13 | 100 |
| 11 | Stroma close to tumor | 1 | 12 | 75 |
| 12 | Stoma > 15 mm from tumor | 1 | 28 | 35.7 |

*55 test samples is less than the potential 68, indicated in Table 1, due to use of 15 samples for training (line 1)

TABLE 3
146 Diagnostic Probe Sets with incidence number greater
than 50 for 150-fold gene selection procedure

| Probe set | Gene symbol | Gene title | LogFC^{#} |
|---|---|---|---|
| 213764_s_at | MFAP5 | microfibrillar associated proteins | −1.73 |
| 209758_s_at | MFAP5 | microfibrillar associated protein 5 | −1.48 |
| 213765_at | MFAP5 | microfibrillar associated protein 5 | −1.36 |
| 210280_at | MPZ | myelin protein zero (Charcot-Marie- | −1.20 |
| | | Tooth neuropathy 1B) | |
| 210198_s_at | PLP1 | proteolipid protein 1 (Pelizaeus- | −1.18 |
| | | Merzbacher disease, | |
| | | spastic paraplegia 2, uncomplicated) | |
| 215104_at | NRIP2 | nuclear receptor interacting | −0.94 |
| | | protein 2 | |
| 213847_at | PRPH | peripherin | −0.93 |
| 214767_s_at | HSPB6 | heat shock protein, alpha- | −0.88 |
| | | crystallin-related, B6 | |
| 209843_s_at | SOX10 | SRY (sex determining | −0.61 |
| | | region Y)-box 10 | |
| 209686_at | S100B | S100 calcium binding protein B | −0.94 |
| 209915_s_at | NRXN1 | neurexin 1 | −0.80 |
| 214023_x_at | TUBB2B | tubulin, beta 2B | −0.75 |
| 214954_at | SUSD5 | sushi domain containing 5 | −0.98 |
| 204584_at | L1CAM | L1 cell adhesion molecule | −1.20 |
| 204777_s_at | MAL | mal, T-cell differentiation protein | −0.99 |
| 205132_at | ACTC1 | actin, alpha, cardiac muscle 1 | −0.99 |
| 203151_at | MAP1A | microtubule-associated protein 1A | −0.69 |
| 210869_s_at | MCAM | melanoma cell adhesion molecule | −0.71 |
| 204627_s_at | ITGB3 | integrrin, beta 3 (platelet | −0.82 |
| | | glycoprotein IIIa, antigen CD61) | |
| 209086_x_at | MCAM | melanoma cell adhesion molecule | −0.61 |
| 219314_s_at | ZNF219 | zinc finger protein 219 | −0.51 |
| 221204_s_at | CRTAC1 | cartilage acidic protein 1 | −0.56 |
| 212886_at | CCDC69 | coiled-coil domain containing 69 | −0.59 |
| 210814_at | TRPC3 | transient receptor potential cation | −0.75 |
| | | channel, subfamily C, member 3 | |
| 212793_at | DAAM2 | dishevelled associated activator | −0.56 |
| | | of morphogenesis 2 | |
| 212565_at | STK38L | serine/threonine kinase 38 like | −0.58 |
| 214606_at | TSPAN2 | tetraspanin 2 | −0.54 |
| 336_at | TBXA2R | thromboxane A2 receptor | −0.65 |
| 218660_at | DYSF | dysferlin, limb girdle muscular | −0.55 |
| | | dystrophy 2B (autosomal recessive) | |
| 214434_at | HSPA12A | heat shock 70 kDa protein 12A | −0.57 |
| 212274_at | LPIN1 | lipin 1 | −0.48 |
| 206874_s_at | — | — | −0.44 |
| 203939_at | NT5E | 5′-nucleotidase, ecto (CD73) | −0.49 |
| 205954_at | RXRG | retinoid X receptor, gamma | −0.53 |
| 219909_at | MMP28 | matrix metallopeptidase 28 | −0.54 |
| 206425_s_at | TRPC3 | transient receptor potential cation | −0.57 |
| | | channel, subfamily C, member 3 | |
| 205433_at | BCHE | butyrylcholinesterase | −0.93 |
| 35846_at | THRA | thyroid hormone receptor, alpha | −0.46 |
| | | (erythroblastic | |
| | | leukemia viral (v-erb-a) | |
| | | oncogene homolog, avian? | |
| 204736_s_at | CSPG4 | chondroitin sulfate proteoglycan 4 | −0.55 |
| 202806_at | DBN1 | drebrin 1 | −0.43 |
| 212097_at | CAV1 | caveolin 1, caveolae protein, | −0.38 |
| | | 22 kDa | |
| 201841_s_at | HSPB1 | heat shock 27 kDa protein 1 | −0.44 |
| 206382_s_at | BDNF | brain-derived neurotrophic factor | −0.62 |
| 219091_s_at | MMRN2 | multimerin 2 | −0.44 |
| 205076_s_at | MTMR11 | myotubularin related protein 11 | −0.57 |
| 204159_at | CDKN2C | cyclin-dependent kinase inhibitor | −0.46 |
| | | 2C (p18, inhibits CDK4) | |
| 212992_at | AHNAK2 | AHNAK nucleoprotein 2 | −0.60 |
| 206024_at | HPD | 4-hydroxyphenylpyruvate | −0.57 |
| | | dioxygenase | |
| 218094_s_at | DBNDD2 /// | dysbindin (dystrobrevin binding | −0.41 |
| | SYSt- | protein 1) domain containing 2 /// | |
| | DBNDD2 | SYS1-DBNDD 2 | |
| 211276_at | TCEAL2 | transcription elongation | −0.52 |
| | | factor A (SII)-like 2 | |
| 209191_at | TUBB6 | tubulin, beta 6 | −0.51 |
| 213675_at | — | CDNA FLJ25106 fis, | −0.44 |
| | | clone CBR01467 | |
| 211340_s_at | MCAM | melanoma cell adhesion molecule | −0.46 |
| 210632_s_at | SGCA | sarcoglycan, alpha | −0.58 |
| | | (50 kDa dystrophin- | |
| | | associated glycoprotein) | |
| 218651_s_at | LARP6 | La ribonucleoprotein | −0.34 |
| | | domain family. member 6 | |
| 207876_s_at | FLNC | filamen C, gamma (actin | −0.45 |
| | | binding protein 280) | |
| 218877_s_at | TRMT11 | tRNA methyltransferase 11 | +0.44 |
| | | homolog (S. cerevisiae) | |
| 219416_at | SCARA3 | scavenger receptor class | −0.57 |
| | | A, member 3 | |
| 209981_at | CSDC2 | cold shock domain containing | −0.56 |
| | | C2, RNA binding | |
| 214212_x_at | FERMT2 | fermitin family homolog | −0.42 |
| | | 2 (Drosophila) | |
| 207554_x_at | TBXA2R | thromboxane A2 receptor | −0.44 |
| 205231_s_at | EPM2A | epilepsy, progressive myoclonus | −0.42 |
| | | type 2A, Lafora disease (laforin) | |
| 215306_at | — | MRNA: cDNA DKFZp586N2020 | −0.48 |
| | | (from clone DKFZp586N2020) | |
| 218435_at | DNAJC15 | DnaJ (Hsp40) homolog, | −0.49 |
| | | subfamily C, member 15 | |
| 203597_s_at | WBP4 | WW domain binding protein | −0.34 |
| | | 4 (formin binding protein 21) | |
| 205303_at | KCNJ8 | potassium inwardly-rectifying | −0.42 |
| | | channel, subfamily 3, member 8 | |
| 201389_at | ITGA5 | integrin, alpha 5 (fibronectin | −0.50 |
| | | receptor, alpha polypeptide) | |
| 204940_at | PLN | phospholamban | −0.49 |
| 220765_s_at | LIMS2 | LIM and senescent cell | −0.41 |
| | | antigen-like domains 2 | |
| 203299_s_at | AP1S2 | adaptor-related protein complex | −0.41 |
| | | 1, sigma 2 subunit | |
| 201344_at | UBE2D2 | ubiquitin-conjugating enzyme | −0.38 |
| | | E2D 2 (UBC4/5 homolog, yeast) | |
| 218648_at | CRTC3 | CREB regulated transcription | −0.33 |
| | | coactivator 3 | |
| 204939_s_at | PLN | phospholamban | −0.45 |
| 201431_s_at | DPYSL3 | dihydropyrimidinase-like 3 | −0.40 |
| 215534_at | — | MRNA; cDNA DKFZp586C1923 | −0.46 |
| | | (from clone DKFZp586C1923) | |
| 209169_at | GPM6B | glycoprotein M6B | −0.34 |
| 209651_at | TGFB1I1 | transforming growth factor beta | −0.42 |
| | | 1 induced transcript 1 | |
| 218711_s_at | SDPR | serum deprivation response | +0.41 |
| | | (phosphatidylserine binding protein) | |
| 212358_at | CLIP3 | CAP-GLY domain containing | −0.47 |
| | | linker protein 3 | |
| 218691_s_at | PDLIM4 | PDZ and LIM domain 4 | −0.42 |
| 218266_s_at | FREQ | frequenin homolog (Drosophila) | −0.46 |
| 210319_x_at | MSX2 | msh homeobox 2 | +0.45 |
| 218545_at | CCDC91 | coiled-coil domain containing 91 | −0.31 |
| 44702_at | SYDE1 | synapse defective 1, Rho GTPase, | −0.38 |
| | | homolog 1 (C. elegans) | |
| 221014_s_at | RAB33B | RAB33B. member RAS | −0.38 |
| | | oncogene family | |
| 221246_x_at | TNS1 | tensin 1 | −0.27 |
| 208789_at | PTRF | polymerase I and transcript | −0.42 |
| | | release factor | |
| 220722_s_at | SLC5A7 | solute carrier family 5 (choline | −0.41 |
| | | transporter), member 7 | |
| 209087_x_at | MCAM | melanoma cell adhesion molecule | −0.40 |
| 221657_s_at | HSPB8 | heat shock 22 kDa protein 8 | −0.40 |
| 205561_at | KCTD17 | potassium channel tetramerisation | −0.32 |
| | | domain containing 17 | |
| 213808_at | — | Clone 23688 mRNA sequence | −0.43 |
| 202565_s_at | SVIL | supervillin | −0.36 |
| 211964_at | C0L4A2 | collagen type IV, alpha 2 | −0.39 |
| 219563_at | C14orf139 | chromosome 14 open | −0.38 |
| | | reading frame 139 | |
| 214122_at | PDLIM7 | PDZ and LIM domain 7 (enigma) | −0.30 |
| 213589_at | RRAS2 | related RAS viral (r-ras) | −0.29 |
| | | oncogene homolog 2 | |
| 205973_at | FEZ 1 | fasciculation and elongation | −0.35 |
| | | protein zeta 1 (zygin I) | |
| 218818_at | FHL3 | four and a half LIM domains 3 | −0.36 |
| 212120_at | RHOQ | ras homolog gene family, member Q | −0.31 |
| 219073_s_at | OSBPL10 | oxysterol binding protein-like 10 | −0.37 |
| 221480_at | HNRNPD | heterogeneous nuclear | −0.36 |
| | | ribonucleoprotein | |
| | | D (AU-rich element | |
| | | RNA binding protein 1, 37 kDa) | |
| 207071_s_at | ACO1 | aconitase 1, soluble | −0.27 |
| 211717_at | ANKRD40 | ankyrin repeat domain 40 | −0.28 |
| 201313_at | ENO2 | enolase 2 (gamma, neuronal) | −0.36 |
| 204628_s_at | ITGB3 | integrin, beta 3 (platelet | −0.31 |
| | | glycoprotein IIIa, antigen CD61) | |
| 204303_s_at | KIAA0427 | KIAA0427 | −0.35 |
| 214439_x_at | BIN1 | bridging intergrator 1 | −0.29 |
| 209015_s_at | DNAJB6 | DnaJ (Hsp40) homolog, subfamily | −0.29 |
| | | B, member 6 | |
| 213547_at | CAND2 | cullin-associated and neddylation- | −0.31 |
| | | dissociated 2 (putative) | |
| 204058_at | ME1 | malic enzyme 1, NADP(+)- | −0.34 |
| | | dependent, cytosolic | |
| 219902_at | BHMT2 | betaine-homocysteine | −0.33 |
| | | methyltransferase 2 | |
| 214306_at | OPA1 | optic atrophy 1 (autosomal | −0.27 |
| | | dominant) | |
| 210201_x_at | BIN1 | bridging integrator 1 | −0.29 |
| 212509_s_at | MXRA7 | matrix-remodelling associated 7 | −0.27 |
| 213231_at | DMWD | dystrophia myotonica, WD | −0.30 |
| | | repeat containing | |
| 201843_s_at | EFEMP1 | EGF-containing fibulin-like | −0.32 |
| | | extracellular matrix protein 1 | |
| 206289_at | HOXA4 | homeobox A4 | −0.29 |
| 203501_at | PGCP | plasma glutamate carboxypeptidase | −0.30 |
| 216894_x_at | CDKN1C | cyclin-dependent kinase inhibitor | −0.27 |
| | | 1C (p57, Kip2) | |
| 216500_at | — | HL14 gene encoding | −0.29 |
| | | beta-galactoside- | |
| | | binding lectin, 3′ end, clone 2 | |
| 220050_at | C9orf9 | chromosome 9 open reading frame 9 | −0.32 |
| 209362_at | MED21 | mediator complex subunit 21 | −0.26 |
| 202931_x_at | BIN1 | bridging integrator 1 | −0.27 |
| 213480_at | VAMP4 | vesicle-associated membrane | −0.24 |
| | | protein 4 | |
| 205611_at | TNFSF12 | tumor necrosis factor (ligand) | −0.29 |
| | | superfamily, member 12 | |
| 204365_s_at | REEP1 | receptor accessory protein 1 | −0.29 |
| 203389_at | KIF3C | kinesin family member 3C | −0.26 |
| 205368_at | FAM131B | family with sequence | −0.27 |
| | | similarity 131, member B | |
| 217066_s_at | DMPK | dystrophia myotonica-protein kinase | −0.29 |
| 212457_at | TFE3 | transcription factor binding | −0.25 |
| | | to IGHM enhancer 3 | |
| 200685_at | SFRS11 | splicing factor, arginine/ | −0.16 |
| | | serine-rich 11 | |
| 200788_s_at | PEA15 | phosphoprotein enriched | −0.22 |
| | | in astrocytes 15 | |
| 202522_at | PITPNB | phosphatidylinositol transfer | −0.16 |
| | | protein beta | |
| 208869_s_at | GABARAPL1 | GABA(A) receptor-associated | −0.19 |
| | | protein like 1 | |
| 209524_at | HDGFRP3 | hepatoma-derrived growth factor, | −0.14 |
| | | related protein 3 | |
| 211347_at | CDC14B | CDC14 cell division cycle 14 | −0.21 |
| | | homolog B (S. cerevisiae) | |
| 211677_x_at | CADM3 | cell adhesion molecule 3 | −0.21 |
| 212610_at | PTPN11 | protein tyrosine phosphatase, | −0.23 |
| | | non-receptor type 11 | |
| | | (Noonan syndrome 1) | |
| 212848_s_at | C9orf3 | chromosome 9 open reading frame 3 | −0.27 |
| 214643_x_at | BIN1 | bridging integrator 1 | −0.23 |
| 217820_s_at | ENAH | enabled homolog (Drosophila) | −0.19 |
| 218597_s_at | CISD1 | CDGSH iron sulfur domain 1 | −0.18 |
| 221502_at | KPNA3 | karyopherin alpha 3 | −0.20 |
| | | (importin alpha 4) | |
| 222221_x_at | EHD1 | EH-domain containing 1 | −0.20 |
| 32625_at | NPR1 | natriuretic peptide | −0.22 |
| | | receptor A/guanylate | |
| | | cyclase A (atrionatriuretic | |
| | | peptide receptor A) | |

LogFC is the logarithm Fold Change as tumorous stroma being compared to normal stroma.
+/− represents up-/down-regulated expression level in tumorous stroma.

D. Testing with Independent Datasets

The classifier then was tested on a number of independent expression microarray Datasets of tumor-bearing tissue, including data from 110 samples generated in this study (Table 2, Datasets 1 & 2) and data from 123 samples generated elsewhere (Table 2, Datasets 3 & 4).

The 131-probe set classifier developed in Example 1C was tested on 243 samples that had not been used for training. Each of these samples contained tumor, though usually very little tumor. Results are shown in Table 2, lines 2 to 5. Using the 131-probe set (114 biomarker) classifier, almost all the 243 samples were recognized as being from cancer patients, with high average accuracy ˜99%. Only two cases were misclassified (marked with an “*” in FIG. 1(a)). Although these samples were ostensibly given tumor percentages of 20% and 25% by pathologists, they were predicted to possibly contain little or no tumor using the CellPred program, which estimates the tissue components using an in silico multiple-variate linear regression model (Wang Y, Xiao-Qin Xia, Zhenyu Jia, Anne Sawyers, Huazhen Yao, Jessica Wang-Rodriquez, Michael McClelland, Dan Mercola, “In silico estimates of tissue components in surgical samples based on expression profiling data,” Cancer Research (2010 Aug. 15; 70(16):6448-55. Epub 2010 Jul. 27 [algorithm available at http://webarraydborg/webarray/indexhtml]). It is possible that these two exceptions were archived incorrectly and are not from patients with cancer or are from a very distant location relative to the tumor. In other words, these samples were predicted to have little or no tumor, although they had been booked as having over 20% tumor, indicating their assignment as tumor may have been a bookkeeping error. Thus, the classifier classified the independent samples as being from cancer patients with an overall accuracy of 98%, a value which compares favorably with the diagnostic accuracy of PSA-based methods of ˜70% (Shariat S F, Scardino P T, Lilja H. Screening for prostate cancer: an update. Can J Urol 2008; 15(6):4363-74).

Correlation of the PAM classification results with cell composition (FIG. 1) was examined. For test cases in Datasets 1 and 2, cell composition values were obtained from pathologists estimates, as described in Example 1A (Materials and Methods).

For Datasets 3 and 4 (FIGS. 1(c) and 1(d) respectively), tumor cell content values were not documented at the time of collection. Accordingly, cell composition was estimated using multigene signatures that are invariant with tumor surgical pathology parameters of Gleason and stage by the CellPRed program (Wang Y, Xiao-Qin Xia, Zhenyu Jia, Anne Sawyers, Huazhen Yao, Jessica Wang-Rodriquez, Michael McClelland, Dan Mercola, “In silico estimates of tissue components in surgical samples based on expression profiling data,” Cancer Research (2010 Aug. 15; 70(16):6448-55. Epub 2010 Jul. 27 [algorithm available at http://webarraydborg/webarray/indexhtml]). Based on the estimations, all 79 samples in Dataset 3 bore tumor, with tumor content ranging from 24% to 87% (FIG. 1(c)); tumor percentage for Dataset 4 ranged from 0% to 80%, FIG. 1(d)).

Based on these tumor percentage data for the various samples shown in FIG. 1, the PAM classification was successful on independent test samples with a broad range of tumor epithelial cells, including samples with just a few percent of epithelial cells. This result demonstrates the accuracy of the classifier in the categorization of prostate cancer cases independent of the presence or amount of the tumor epithelial component.

The classifier was then tested using specimens composed of normal prostate stroma and epithelium (Table 2, lines 6-7). Twelve biopsies from the DFMO study, all of them different from the 15 samples used earlier for training, were separated into two groups. In group (1) were seven second biopsies, taken twelve months later, from the same participants whose first biopsy samples were included in the training set. These samples were accurately (100%) identified as nontumor (Table 2, line 6). In group (2) were five biopsy samples not from subjects previously used for training. Two out of these 5 biopsy samples were categorized as being from cancer patients (Table 2 line 7). When the histories for these volunteers were investigated it was found that both donors had consistently exhibited elevated PSA levels of 6.1 and 8 ng/ml, (normal values <3 ng/ml) respectively although no tumor was observed in either of two sets of sextant biopsies obtained from these volunteers. The volunteers also had a history of prostate cancer in the family. All other donors of the normal biopsy volunteers exhibited normal PSA values. The IRB-approved protocol precluded following up further to establish that these patients had cancer that had been missed in the biopsies.

The classifier was then tested on 13 specimens obtained by rapid autopsy of individuals dying of unrelated causes (Table 2, line 8). Twelve out of 13 of these samples, 92% accuracy, were classified as nontumor. Histological examination of all embedded tissue (multiple blocks of the glands taken from both lobes and all zones) of the one potentially “misclassified” case revealed tumor foci (multiple foci of small “latent” tumors). Thus, the potential “misclassifications” correlated well with unusual clinical and pathological features of the cases. In summary, the handful of potential misclassifications were on samples for which there was evidence of mislabeling before the test, indicating a higher sensitivity/specificity of perhaps 100%.

In summary, 25 nominally normal samples were classified as being from donors without prostate cancer, or were classified in accordance with abnormal features that were subsequently uncovered. These results demonstrate the ability of the classifier to discriminate among normal and abnormal prostate tissue in the absence of histological recognizable tumor cells in the samples studied.

E. Validation of the classifier: manual microdissection, random classifiers and published literature.

Histologically confirmed samples of stroma adjacent to tumor were developed for validation of the classifier. 153 samples from datasets 1 and 4 were used to prepare “pure” stroma samples (i) adjacent, (ii) close, and (iii) far (>15 mm) from known tumor foci. Specifically, an etching procedure was used to prepare 71 samples of tumor-adjacent stroma from patient tissues of Dataset 2, and thirteen samples from Dataset 4. An additional twelve samples from Dataset 1 were obtained from OCT blocks entirely by manual microdissection, i.e. without etching but leaving a margin of tissue between tumor and stroma, followed by histologically examined by frozen section analysis of the OCT surface and bottom side of the pieces, to ensure the absence of tumor. These twelve manually excised pieces were termed “close stroma” (˜3 mm). The expression values for all 96 samples were used to test the 131 probe set classifier using the PAM procedure. As summarized in Table 2, lines 9-11, samples were classified as from tumor-bearing patients with an accuracy of 97% for the 71 adjacent stroma samples from dataset 2, 100% for thirteen adjacent stroma samples from dataset 4, and 75% for the twelve “close stroma” samples from dataset 1, representing an overall accuracy of 95% for the 96 independent samples.

Five of the 96 samples appeared potentially “misclassified” as normal. Three of these potential misclassifications represented samples among the twelve close stroma samples from Dataset 1. Because these samples were obtained by manual excision, some may not have been as near to tumor as the samples obtained by the etching method.

To evaluate the distance relative to the tumor at which the expression changes characteristics of tumor stroma extended, twenty-eight (28) samples greater than 15 mm from any known tumor were obtained and analyzed using the classifier. The samples were generally from the contralateral lobe. The results are presented in Table 2, line 12. Only ten of the 28 samples (36%) were categorized as tumor-associated stroma. Using the Fisher Exact Test, the distribution for the 28 “remote” samples was significantly different from the 12 stroma samples from “close” to tumor of the same patient tissues (p value=0.038).

Thus, the presence of tumor in the prostate was detected in tumor-adjacent, close, and far (>15 mm) samples with a decreasing accuracy of 98%, 75% and 36%, respectively. This result shows that in this study, the expression changes recognized by the classifier declined with increasing distance of stroma from tumor. This observation of a gradual reduction in the sensitivity of the classifier as the distance from tumor increases bears on the likely mechanism for the production of differential gene expression in tumor adjacent stroma which is generally believed to involve the influence of “paracrine” factors emanating from tumor foci (Cunha G R, Hayward S W, Wang Y Z, Ricke W A. “Role of the stromal microenvironment in carcinogenesis of the prostate.” Int J Cancer 2003; 107(1):1-10; Tuxhorn J A, Ayala G E, Rowley D R. “Reactive stroma in prostate cancer progression,” J Urol 2001; 166(6):2472-83; Rowley D R. “What might a stromal response mean to prostate cancer progression?” Cancer Metastasis Rev 1998; 17(4):411-9).

Indeed the tumor microenvironment is likely the source of factors that are required for tumor formation by the epithelial component (Cunha G R, Hayward S W, Wang Y Z, Ricke W A., “Role of the stromal microenvironment in carcinogenesis of the prostate,” Int J Cancer 2003; 107(1):1-10). The number of diffusible paracrine factors of this complex interaction mechanism likely declines with separation of target cells from the secreting cells. A simple radial dilution model would predict a decline of effects of tumor-derived factors by at least the square of the distance of target stroma cells from a tumor focus. Based on this simple model, the decrease in the frequency of categorization stroma taken from over 15 mm from a known tumor focus to 36% suggests a 50% recognition distance of ˜13 mm in fresh frozen tissue. In view of the relatively modest average fold-change of the 131 probe sets of the classifier (see Table 3) the distance at which “presence-of-tumor” is recognized indicates a surprisingly large range of “influence” of tumor over steady state gene expression changes in nearby stroma.

Cunha G R, Hayward S W, Wang Y Z, Ricke W A. Role of the stromal microenvironment in carcinogenesis of the prostate. Int J Cancer 2003; 107(1):1-10

Normal samples and rapid autopsy samples could be easily distinguished from samples containing tumor using many of the individual genes (e.g., heatmap, FIG. 5). Differences distinguishing near stroma from control stroma can be more subtle and vary between patients; this distinction can require a classifier based on a number of genes.

Further validation included a comparison with 100 random classifiers generated by arbitrarily sampling 131 probe sets for each classifier. 100 randomized experiments were carried out using the 22,277 probe sets of the U133A platform used for the original 13 tumor-bearing training cases. In each experiment 2,210 probe sets were randomly selected from the 12,901 probe sets remaining after subtracting 9376 aging-related probe sets from the 22,277 probe sets. The remaining probe sets were screened with the same MLR criteria used for the development of the 131-probe set classifier described in Example 1A (Materials and Methods). The genes that survived MLR filter were used to develop a classifier with PAM exactly as for the 131-probe set classifier. PAM selected an average of 6.2 (standard deviation=2.3) probe sets (<<131) and the average performance of these random-gene classifiers based on the tests of other datasets is summarized in Table 4.

TABLE 4
Comparison of 131-probe set classifier to classifiers generated from “random” genes

| | | | | Accuracy | | Sensitivity | | Specificity | |
|---|---|---|---|---|---|---|---|---|---|
| | | | Case | (%) | | (%) | | (%) | |
| | | Dataset | No. | i | ii | i | ii | i | ii |
| 1 | Training set | 1 | 26 | 96.4 | 67.1 | 92.3 | 32.5 | 100 | 97.1 |
| | | | (13 + 13) | | | | | | |

Test set
Tumor

| | | | | | | | | | |
|---|---|---|---|---|---|---|---|---|---|
| 2 | Tumor-bearing | 1 | 55 | 100 | 8.7 | 96.4 | 8.7 | NA | NA |
| | | | (68 − 13) | | | | | | |
| 3 | Tumor-bearing | 2 | 65 | 100 | 12.9 | 100 | 12.9 | NA | NA |
| 4 | Tumor-bearing | 3 | 79 | 100 | 13.4. | 100 | 13.4 | NA | NA |
| 5 | Tumor-bearing | 4 | 44 | 100 | 15.9 | 100 | 15.9 | NA | NA |

Normal

| | | | | | | | | | |
|---|---|---|---|---|---|---|---|---|---|
| 6 | Biopsies (1) | 1 | 7 | 100 | 98.8 | NA | NA | 100 | 98.8 |
| 7 | Biopsies (2) | 1 | 5 | 60 | 100 | NA | NA | 60.0 | 100 |
| 8 | Rapid autopsies | 1 | 13 | 92.3 | 67.5 | NA | NA | 92.3 | 67.5 |

Manual Microdissected /LCM

| | | | | | | | | | |
|---|---|---|---|---|---|---|---|---|---|
| 9 | Tumor-adjacent | 2 | 71 | 97.1 | 13.6 | 97.1 | 13.6 | NA | NA |
| | stroma | | | | | | | | |
| 10 | Tumor-adjacent | 4 | 13 | 100 | 15.9 | 100 | 15.9 | NA | NA |
| | stroma | | | | | | | | |
| 11 | Tumor-adjacent | 1 | 12 | 75.0 | 5.8 | 75.0 | 5.8 | NA | NA |
| | stroma | | | | | | | | |
| 12 | Tumor-bearing | 5 | 12 | 100 | 19.2 | 100 | 19.2 | NA | NA |
| 13 | Pooled normal | 5 | 4 | 100 | 79.4 | NA | NA | 100 | 79.4 |
| | stroma | | | | | | | | |

The random classifiers were biased towards calling almost all samples as being normal, leading to a statistically significant under-calling of sets of tumor samples, (e.g. Table 4, line 2) and a statistically significant “success” in calling normal samples (e.g. Table 4, lines 6-8, 13). The overall accuracy was around 35%, no different from random accuracy. Thus, these random classifiers had no diagnostic value, further demonstrating that the results obtained with the 131-probe set classifier cannot be attributed to chance.

The fact that representative genes were in fact preferentially expressed in stroma was validated by PCR, and independent cases from a formalin-fixed and paraffin-embedded (FFPE) clinical collection used to verify translational relevance. Gene expression was assessed by a modified quantitative PCR procedure, as described in Example 1A (Materials and Methods). In a limited survey, four genes were found to have reliably preserved short amplicons. Blocks of sixty three tumor cases were examined and tumor and stroma regions in H & E sections were demarcated by a pathologist (DAM). Punches were removed from adjacent unstained sections and used for PCR for 63 tumor portions and 38 stroma portions. For all four genes, highly significant preferential expression in stroma was observed. These results for independent cases and by an independent method further support the preferential expression of these genes in tumor stroma and further argue that the classifier may be adapted to clinical biopsies preserved in FFPE, the standard method of archiving patient biopsies.

Two studies reporting expression analysis results for subclasses of the stroma of prostate cancer (Richardson A M, Woodson K, Wang Y, et al., “Global expression analysis of prostate cancer-associated stroma and epithelia,” Diagn Mol Pathol 2007; 16(4):189-97; Dakhova O, Ozen M, Creighton C J, et al., “Global gene expression analysis of reactive stroma in prostate cancer,” Clin Cancer Res 2009; 15(12):3979-89) describing expression analysis for subclasses of prostate stroma showed consistent findings.

One study (In one study (Richardson A M, Woodson K, Wang Y, et al., “Global expression analysis of prostate cancer-associated stroma and epithelia,” Diagn Mol Pathol 2007; 16(4):189-97;) identified 44 (39 unique) genes as differentially expressed between intratumor stroma and normal stroma using Affymetrix U133Plus2.0 GeneChips on five paired LCM intratumor stroma and matched normal stroma. The microarray data from this study are not publicly available; a detailed comparison is not possible. Several of the 44 genes were recognized as differentially expressed in our analysis; however, none survived the age and tumor epithelial cell expression filters applied here.

Another study (Dakhova O, Ozen M, Creighton C J, et al., “Global gene expression analysis of reactive stroma in prostate cancer,” Clin Cancer Res 2009; 15(12):3979-89) used Agilent 44K gene expression arrays on 17 paired laser-captured microdissected (LCM) reactive stroma samples and matched normal stroma samples. 1,141 genes were identified as differentially expressed between “reactive” and normal stroma. Reactive stroma has been studied in detail (Dakhova O, Ozen M, Creighton C J, et al., “Global gene expression analysis of reactive stroma in prostate cancer,” Clin Cancer Res 2009; 15(12):3979-89; Richardson A M, Woodson K, Wang Y, et al., “Global expression analysis of prostate cancer-associated stroma and epithelia,” Diagn Mol Pathol 2007; 16(4):189-97) is a form of stroma very near to tumors which differs from normal stroma in histological appearance, cell composition and gene and protein expression. Prostate cancer cases with defined reactive stroma exhibit a significantly decreased postprostatectomy disease-free survival (Ayala G, Tuxhorn J A, Wheeler T M, et al, “Reactive stroma as a predictor of biochemicalfree recurrence in prostate cancer,” Clin Cancer Res 9:4792-801, 2003a).

To assess similarities of the 339 probe set basis set with the data of Dakhova et al., the raw microarray Dataset was downloaded from public Gene Expression Omnibus database (Accession SE11682). t tests were used for differential analysis and 2967 genes (6.6% of the 45015 genes on the array) were identified as differentially expressed with a p cutoff of 0.05. This loose criterion was used to generate a relatively larger gene list to be compared to the classifier basis set described in this example of 339 probe sets. The 339 probe sets were mapped to 557 genes on the Agilent array. 38 of the genes were among the 2967 Agilent genes that exhibited significant differential expression (p<0.05). Thirty one of these 38 genes sowed concordance in differential expression between the two studies (Table 5). Additional similarities were likely to have been masked by platform-specific effects (Affymetrix versus Agilent).

TABLE 5
Concordence of 38 overlapping genes/probe sets of the 339
probe sets of the diagnostic classifier with
the sign of differential change of Dakhova et al.

| Attymetrix | | | | |
|---|---|---|---|---|
| Probe Set | | Gene | Thio | Dakova |
| ID | Agilent Probe ID | Symbol | Study | et al. |
| 205554_s_at | 25330 | DNABE1L3 | up | up |
| 207332_s_at | 6474 | TFRC | up | up |
| 207332_s_at | 33074 | TFRC | up | down |
| 205765_at | 27257 | ADAM19 | down | up |
| 206331_at | 41622 | CALCRL | down | up |
| 201655_s_at | 15643 | HPB32 | down | up |
| 207437_at | 22289 | NOVA1 | down | up |
| 205554_at | 40101 | RXRG | down | up |
| 210432_s_at | 19493 | SCN3A | down | up |
| 215502_at | 10102 | BHMT2 | down | down |
| 212097_at | 5648 | CAV1 | down | down |
| 212097_at | 6348 | CAV1 | down | down |
| 212097_at | 40981 | CAV1 | down | down |
| 208792_s_at | 32464 | CLU | down | down |
| 213428_s_at | 21788 | CDL5A1 | down | down |
| 205015_s_at | 1064 | DNAJB6 | down | down |
| 218435_at | 12280 | DNAJC15 | down | down |
| 204410_at | 32431 | EIF1AY | down | down |
| 207876_s_at | 6002 | FLNC | down | down |
| 205674_x_at | 29788 | FXYD2 | down | down |
| 211275_s_at | 43496 | GYG1 | down | down |
| 205561_at | 22259 | KCTD17 | down | down |
| 216056_s_at | 24526 | NRXN1 | down | down |
| 205515_s_at | 24526 | NRXN1 | down | down |
| 204540_at | 32154 | FLN | down | down |
| 204539_s_at | 32154 | FLN | down | down |
| 203466_at | 43556 | PRAF2 | down | down |
| 208131_s_at | 2097 | PTGIB | down | down |
| 212610_at | 38709 | PTPN11 | down | down |
| 208769_at | 13320 | PTRF | down | down |
| 212887_at | 44512 | SEC23A | down | down |
| 201312_s_at | 38622 | SH35GRL | down | down |
| 213203_at | 12610 | SNAPC5 | down | down |
| 213203_at | 26477 | SNAPC5 | down | down |
| 216087_s_at | 5944 | SORBB1 | down | down |
| 202440_s_at | 5316 | ST5 | down | down |
| 212457_at | 2313 | TFE3 | down | down |
| 213460_at | 41809 | VAMP4 | down | down |

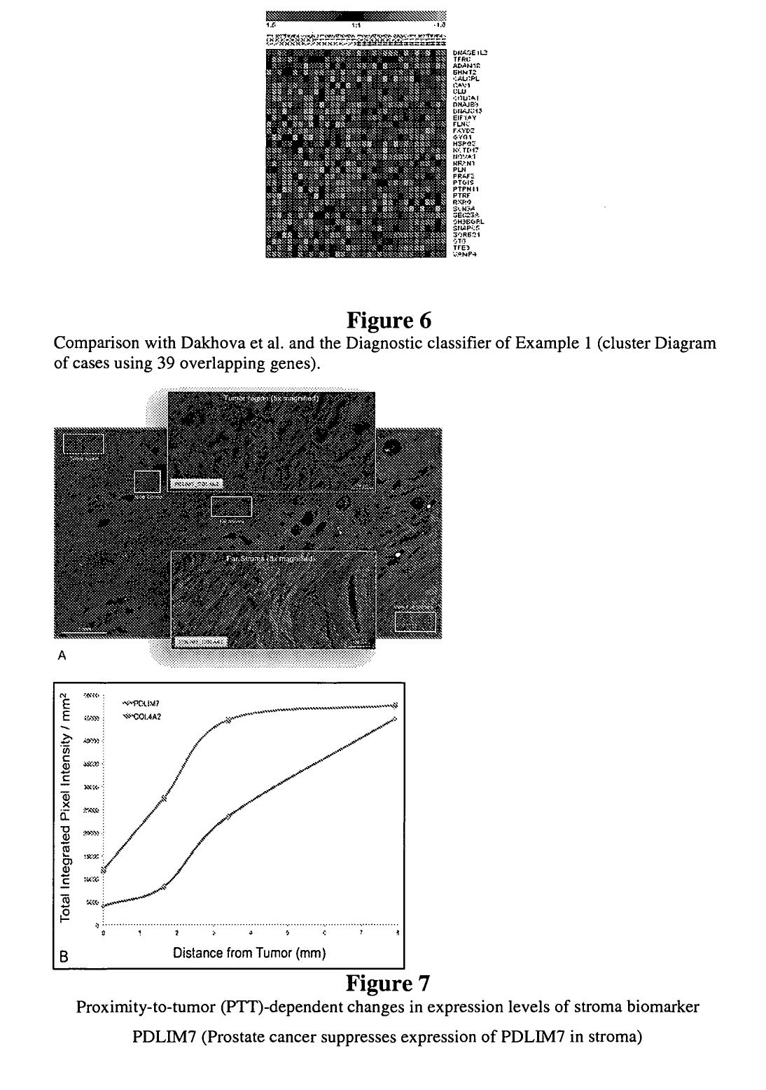

This overlap of 31 concordant genes between the two lists of 339 and 557 genes exceeds that expected by chance alone (p=0.0001, see Table 5). These genes alone successfully categorize the cases of Dakhova et al. 5 into reactive and normal stroma cases (FIG. 6, showing a heatmap made for the 38 genes using the Aligent microarray data set; the top two genes identified as up-regulated in the study described here; the bottom 30 genes were down-regulation (see comparison presented in Table 5, above)).

The significance of the 38-31-gene concordance was assessed by comparison to a random model with using simulation, where “38” denoted the number of common genes in terms of gene identity between two studies while “31” indicated how many genes out of these “38” identity-concordant genes also concur on alteration tendency. 557 probe sets were randomly, independently and respectively selected and 2,502 probe sets derived above from the total of 37,765 probe sets of Agilent basis. If the number ofcommon probe sets between these two sets of randomly selected genes is equal to or greater than 38 and no less than 31 of these identity-concordant probe sets have similar alteration direction, we increased the simulation count, C, by 1. We repeated this process by 10000 times. The p values associated with this test is defined as C/10000. In this simulation study, the p value was 0.003, indicating that the observed 38 overlapping probe sets can not be explained by chance and therefore our study and that of Dakhova et al. are independent studies that are mutually supportive (an an algorithm in R is available at http://www.pathology.uci.edu/faculty/mercola/UCISPECSHome.html).

The differences in the genes identified in the two studies may be at least partly explained by the fact that the study described in this Example was designed to identify as many changes as possible that were common to all stroma in the presence of tumor. In contrast Dakhova et al. used only “reactive” stroma as defined by the Masson's trichrome staining pattern.

Tumors exhibiting the reactive stroma pattern have been associated with poor postprostatectomy disease-free survival (Yanagisawa N, Li R, Rowley D, et al, “Stromogenic prostatic carcinoma pattern (carcinomas with reactive stromal grade 3) in needle biopsies predicts biochemical recurrence-free survival in patients after radical prostatectomy,” Hum Pathol 38:1611-20, 2007). The overlap in the lists may include expression changes that occur in reactive stroma, thereby strengthening the PAM classifier for samples near the poor prognosis tumors. Thus, the overlapping genes of the Diagnostic Classifier described in this Example also have prognostic significance (FIG. 6).

Thus, the 339 probe sets (Affymetrix arrays) identified in this study map to 557 genes on Agilent arrays which have been used for deriving profiles for “reactive” stroma, a special case of adjacent stroma associated with poor outcome disease (Dakhova et al.). A total of 31 genes or probe sets appeared to be concordant (in terms of gene identity and the direction of expression alteration) between the 339 probe sets (Affymetrix arrays) identified in this study and the 557 mapped genes (Agilent arrays) in the “reactive” stroma study (Dakhova et al.) with P value=0.0001 (Table 5). The formation of this stroma in prostate cancer has been associated with poor prognosis, suggesting that given that reactive stroma has been associated with poor prognosis. Thus, the diagnostic markers in stroma also are of prognostic interest.

In summary, expression profiles of 13 biopsies containing stroma near tumor and 15 biopsies from volunteers without prostate cancer. About 3,800 significant expression changes were found and thereafter filtered using independent expression profiles to eliminate possible age-related genes and genes expressed at detectable levels in tumor cells. A stroma-specific classifier for nearby tumor was constructed based on 114 candidate genes and tested on 364 independent samples, including 243 tumor-bearing samples and 121 non-tumor samples (normal biopsies, normal autopsies, remote stroma, as well as stroma within a few millimeters of tumor). The classifier predicted the tumor status of patients using tumor-free samples with an average accuracy of 97.2% (sensitivity=97.9% and specificity=88.0%) whereas classifiers trained with sets of 100 randomly generated genes had no diagnostic value. These results demonstrate that the prostate cancer microenvironment exhibits reproducible changes useful for categorizing the presence of tumor in patients when a prostate sample is derived from near the tumor but does not contain any recognizable tumor. The results further demonstrate that the diagnostic changes in the stroma are dependent on the proximity of the stroma to tumor, indicating a gradient in the response to tumor depending on distance.

The classifier developed here used highly selective methods to enrich for mesodermal and ectodermal derivatives compared to endoderm/epithelial derivatives. Computer assisted gene enrichment analysis classification using DAVID (Dennis G, Jr., Sherman B T, Hosack D A, et al. “DAVID: Database for Annotation, Visualization, and Integrated Discovery,” Genome Biol 2003; 4(5):P3) identified a number of statistically significant gene enrichment categories. The 10 most significant are summarized in Table 6.

Numerous genes associated with expression in nerve and muscle are apparent, such as the nine genes of the actin cytoskeleton enrichment category, and in the disease mutation category, including MPZ (Charcot-Maire-Tooth neuropathy 1b), optic atrophy 1, EPM2a (Lafora Disease), BDGF, PLN (phospholamban), SGCA (dystophin-associated glycoprotein), and EFEMP. Biochemical associations include genes related to the TGFβ pathway (SMAD3, TGFIT, ID4, CKDN1C/p57), the Wnt pathway (FZD7, SMAD3, DAAM1 and WISP2) and interacting genes (PCH12, PCDH7, CDH19). These pathways are associated with tumor-stroma paracrine interactions (Richardson A M, Woodson K, Wang Y, et al., “Global expression analysis of prostate cancer-associated stroma and epithelia,” Diagn Mol Pathol 2007; 16(4):189-97; Dakhova O, Ozen M, Creighton C J, et al., “Global gene expression analysis of reactive stroma in prostate cancer,” Clin Cancer Res 2009; 15(12):3979-89; Yanagisawa N, Li R, Rowley D, et al. “Stromogenic prostatic carcinoma pattern (carcinomas with reactive stromal grade 3) in needle biopsies predicts biochemical recurrence free survival in patients after radical prostatectomy,” Hum Pathol 2007; 38(11):1611-20; Tuxhorn J A, McAlhany S J, Yang F, Dang T D, Rowley D R. “Inhibition of transforming growth factor-beta activity decreases angiogenesis in a human prostate cancer-reactive stroma xenograft model.” Cancer Res 2002; 62(21):6021-5; Zhang Q, Helfand B T, Jang T L, et al. “Nuclear factor-kappaB-mediated transforming growth factor-beta-induced expression of vimentin is an independent predictor of biochemical recurrence after radical prostatectomy,” Clin Cancer Res 2009; 15(10):3557-67).

Thus, this study shows that the identified diagnostic changes in stroma are dependent on the proximity of stroma to tumor, indicating a gradient in response to tumor depending on distance.)

Given that reactive stroma has been associated with poor prognosis (Yanagisawa N, Li R, Rowley D, et al. “Stromogenic prostatic carcinoma pattern (carcinomas with reactive stromal grade 3) in needle biopsies predicts biochemical recurrence free survival in patients after radical prostatectomy,” Hum Pathol 2007; 38(11):1611-20), some of the 131 diagnostic markers identified in stroma are of prognostic interest. The classifier may further identify other prostate lesions, including acute and chronic inflammation of the prostate.

TABLE 6
Function Enrichment Analysis

| | | |
|---|---|---|
| Category | AFFY_ID | Gene Name |

Anatomical structure
development

| | | |
|---|---|---|
| 1 | 206874_s_at | collagen, type xvii, alpha 1 |
| 2 | 205303_at | potassium inwardly-rectifying channel, |
| | | subfamily j, member 8 |
| 3 | 209915_s_at | neurexin 1 |
| 4 | 205973_at | fasciculation and elongation protein |
| | | zeta 1 (zygin i) |
| 5 | 210198_s_at | proteolipid protein 1 (pelizaeus-merzbacher |
| | | disease, spastic parapleg 2, uncomplicated) |
| 6 | 205611_at | tumor necrosis factor (ligand) |
| | | superfamily, member 12 |
| 7 | 206289_at | homeobox a4 |
| 8 | 218818_at | four and a half lim domains 3 |
| 9 | 210280_at | myelin protein zero (charcot-marie- |
| | | tooth neuropathy 1b) |
| 10 | 214023_x_at | tubulin, beta 2b |
| 11 | 210632_s_at | sarcoglycan, alpha (50 kda dystrophin- |
| | | associated glycoprotein) |
| 12 | 216894_x_at | cyclin-dependent kinase inhibitor 1c (p57, kip2) |
| 13 | 212457_at | transcription factor binding to ighm enhancer 3 |
| 14 | 213808_at | adam metallopeptidase domain 23 |
| 15 | 201431_s_at | dihydropyrimidinase-like 3 |
| 16 | 214122_at | pdz and lim domain 7 (enigma) |
| 17 | 215306_at | luteinizing hormone/choriogonadotropin receptor |
| 18 | 202565_s_at | supervillin |
| 19 | 212120_at | ras homolog gene family, member q |
| 20 | 211964_at | collagen, type iv, alpha 2 |
| 21 | 205132_at | actin, alpha, cardiac muscle |
| 22 | 210869_s_at | melanoma cell adhesion molecule |
| | 209086_x_at | |
| | 211340_s_at | |
| | 209087_x_at | |
| 23 | 209169_at | glycoprotein m6b |
| 24 | 204736_s_at | chondroitin sulfate proteoglycan |
| | | 4 (melanoma-associated) |
| 25 | 204777_s_at | mal, t-cell differentiation protein |
| 26 | 209686_at | s100 calcium binding protein, beta (neural) |
| 27 | 214212_x_at | pleckstrin homology domain containing, |
| | | family c (with ferm domain member 1 |
| 28 | 216500_at | lectin, galactoside-binding, soluble, 1 (galectin 1) |
| 29 | 210319_x_at | msh homeobox homolog 2 (drosophila) |
| 30 | 212097_at | caveolin 1, caveolae protein, 22 kda |
| 31 | 206382_s_at | brain-derived neurotrophic factor |
| 32 | 204159_at | cyclin-dependent kinase inhibitor 2c |
| | | (p18, inhibits cdk4) |
| 33 | 204939_s_at | phospholamban |
| | 204940_at | |
| 34 | 209843_s_at | sry (sex determining region y)-box 10 |
| 35 | 202806_at | drebrin 1 |
| 36 | 204584_at | 11 cell adhesim molecule |

System development

| | | |
|---|---|---|
| 1 | 206874_s_at | collagen, type xvii, alpha 1 |
| 2 | 205303_at | potassium inwardly-rectifying channel, |
| | | subfamily j, member 8 |
| 3 | 209915_s_at | neurexin 1 |
| 4 | 205973_at | fasciculation and elongation protein |
| | | zeta 1 (zygin i) |
| 5 | 210198_s_at | proteolipid protein 1 (pelizaeus-merzbacher |
| | | disease, spastic paraplegia 2, uncomplicated) |
| 6 | 205611_at | tumor necrosis factor (ligand) |
| | | superfamily, member 12 |
| 7 | 218818_at | four and a half lim domains 3 |
| 8 | 210280_at | myelin protein zero (charcot-marie- |
| | | tooth neuropathy 1b) |
| 9 | 214023_x_at | tubulin, beta 2b |
| 10 | 210632_s_at | sarcoglycan, alpha (50 kda |
| | | dystrophin-associated glycoprotein) |
| 11 | 216894_x_at | cyclin-dependent kinase inhibitor 1c (p57. kip2) |
| 12 | 212457_at | transcription factor binding to ighm enhancer 3 |
| 13 | 213808_at | adam metallopeptidase domain 23 |
| 14 | 201431_s_at | dihydropyrimidinase-like 3 |
| 15 | 214122_at | pdz and lim domain 7 (enigma) |
| 16 | 215305_at | luteinizing homone/choriogonadotropin receptor |
| 17 | 202565_s_at | supervillin |
| 18 | 211964_at | collagen, type iv, alpha 2 |
| 19 | 205132_at | actin, alpha, cardiac muscle |
| 20 | 209163_at | glycoprotein m6b |
| 21 | 204736_s_at | chondroitin sulfate proteoglycan |
| | | 4 (melanoma-associated) |
| 22 | 204777_s_at | mal, t-cell differentiation protein |
| 23 | 209686_at | s100 calcium binding protein, beta (neural) |
| 24 | 216500_at | lectin, galactoside-binding, |
| | | soluble. 1 (galectin 1) |
| 25 | 210319_x_at | msh homeobox homnolog 2 (drosophila) |
| 26 | 206382_s_at | brain-derived neurotrophic factor |
| 27 | 204159_at | cyclin-dependent kinase inhibitor |
| | | 2c (p18. inhibits cdk4) |
| 28 | 204939_s_at | phospholamban |
| | 204940_at | |
| 29 | 202806_at | drebrin 1 |
| 30 | 204584_at | 11 cell adhesion molecule |

Developmental process

| | | |
|---|---|---|
| 1 | 206874_s_at | collagen, type xvii, alpha 1 |
| 2 | 209015_s_at | dnaj (hsp40) homolog, subfamily b, member 6 |
| 3 | 205303_at | potassium inwardly-rectifying |
| | | channel, subfamily j, member 8 |
| 4 | 209915_s_at | neurexin 1 |
| 5 | 205973_at | fasciculation and elongation protein |
| | | zeta 1 (zygin i) |
| 6 | 205611_at | tumor necrosis factor (ligand) |
| | | superfamily, member 12 |
| 7 | 210198_s_at | proteolipid protein 1 (pelizaeus-merzbacher |
| | | disease, spastic paraplegia 2, uncomplicated) |
| 8 | 206289_at | homeobox a4 |
| 9 | 218818_at | four and a half lim domains 3 |
| 10 | 212274_at | lipin 1 |
| 11 | 210280_at | myelin protein zero (charcot-marie- |
| | | tooth neuropathy 1b) |
| 12 | 202931_x_at | bridging integrator 1 |
| | 210201_x_at | |
| | 214439_x_at | |
| 13 | 214023_x_at | tubulin, beta 2b |
| 14 | 201841_s_at | heat shock 27 kda protein 1 |
| 15 | 210632_s_at | sarcoglycan alpha (50 kda dystrophin- |
| | | associated glycoprotein) |
| 16 | 214306_at | optic atrophy 1 (autosomal dominant) |
| 17 | 216894_x_at | cychn-dependent kinase inhibitor 1c (p57, kip2) |
| 18 | 212457_at | transcription factor binding to ighm enhancer 3 |
| 19 | 213808_at | adam metallopeptidase domain 23 |
| 20 | 214122_at | pdz and lim domain 7 (enigma) |
| 21 | 201431_s_at | dihydropyrimidinase-like 3 |
| 22 | 215306_at | luteinizing hormone/choriogonadotropin receptor |
| 23 | 202565_s_at | supervillin |
| 24 | 212120_at | ras homolog gene family, member q |
| 25 | 211964_at | collagen, type iv, alpha 2 |
| 26 | 205132_at | actin, alpha, cardiac muscle |
| 27 | 210869_s_at | melanoma cell adhesion molecule |
| | 209086_x_at | |
| | 211340_s_at | |
| | 209087_x_at | |
| 28 | 204628_s_at | integrin, beta 3 (platelet glycoprotein |
| | | iiia, antigen cd61) |
| | 204627_s_at | |
| 29 | 204736_s_at | chondroitin sulfate proteoglycan |
| | | 4 (melanoma-associated) |
| 30 | 209169_at | glycoprotein m6b |
| 31 | 204777_s_at | mal, t-cell differentiation protein |
| 32 | 209686_at | s100 calcium binding protein, beta (neural) |
| 33 | 214212_x_at | pleckstrin homology domain containing, |
| | | family c (with ferm domain) member 1 |
| 34 | 216500_at | lectin, galactoside-binding, soluble, 1 (galectin 1) |
| 35 | 210319_x_at | msh homeobox homolog 2 (drosophila) |
| 36 | 212097_at | caveohn 1, caveolae protein, 22 kda |
| 37 | 206382_s_at | brain-derived neurotrophic factor |
| 38 | 209651_at | transforming growth factor beta 1 |
| | | induced transcript 1 |
| 39 | 204159_at | cyclin-dependent kinase inhibitor |
| | | 2c (p18, inhibits cdk4) |
| 40 | 204939_s_at | phospholamban |
| | 204940_at | |
| 41 | 209843_s_at | sry (sex determining region y)-box 10 |
| 42 | 202306_at | drebrin 1 |
| 43 | 206874_s_at | ste20-like kinase (yeast) |
| 44 | 204584_at | 11 cell adhesion molecule |

Disease mutation

| | | |
|---|---|---|
| 1 | 221667_s_at | heat shock 22 kda protein 8 |
| 2 | 206874_s_at | collagen, type xvii, alpha 1 |
| 3 | 210198_s_at | proteolipid protein 1 (pelizaeus-merzbacher |
| | | disease, spastic paraplegia 2, uncomplicated) |
| 4 | 206024_at | 4-hydroxyphenylpyruvate dioxygenase |
| 5 | 207554_x_at | thromboxane a2 receptor |
| | 336_at | |
| 6 | 202931_x_at | bridging integrator 1 |
| | 210201_x_at | |
| | 214439_x_at | |
| 7 | 210230_at | myelin protein zero (charcot-marie- |
| | | tooth neuropathy 1b) |
| 8 | 201841_s_at | heat shock 27 kda protein 1 |
| 9 | 210632_s_at | sarcoglycan, alpha (50 kda |
| | | dystrophin-associated glycoprotein) |
| 10 | 214306_at | optic atrophy 1 (autosomal dominant) |
| 11 | 216894_x_at | cyclin-dependent kinase inhibitor 1c (p57, kip2) |
| 12 | 205433_at | butyrylcholinesterase |
| 13 | 215306_at | luteinizing hormone/choriogonadotropin receptor |
| 14 | 213660_at | dysferlin, limb girdle muscular dystrophy |
| | | 2b (autosomal recessive) |
| 15 | 205132_at | actin, alpha, cardiac muscle |
| 16 | 201843_s_at | egf-containing fibulin-like extracellular |
| | | matrix protein 1 |
| 17 | 204628_s_at | integrin, beta 3 (platelet glycoprotein |
| | | iiia, antigen cd61) |
| | 204627_s_at | |
| 18 | 205231_s_at | epilepsy, progressive myoclonus |
| | | type 2a, lafora disease (laforin) |
| 19 | 204365_s_at | receptor accessory protein 1 |
| 20 | 210319_x_at | msh homeobox homolog 2 (drosophila) |
| 21 | 212097_at | caveolin 1, caveolae protein, 22 kda |
| 22 | 206382_s_at | brain-derived neurotrophic factor |
| 23 | 204159_at | cyclin-dependent kinase inhibitor |
| | | 2c (p18, inhibits cdk4) |
| 24 | 204939_s_at | phospholamban |
| | 204940_at | |
| 25 | 209643_s_at | sry (sex determining region y)-box 10 |
| 26 | 204584_at | 11 cell adhesion molecule |

Multicellular organismal
development

| | | |
|---|---|---|
| 1 | 206874_s_at | collagen, type xvii, alpha 1 |
| 2 | 205303_at | potassium inwardly-recfifying |
| | | channel, subfamily j, member 8 |
| 3 | 209915_s_at | neurexin 1 |
| 4 | 205973_at | fasciculation and elongation protein |
| | | zeta 1 (zygin i) |
| 5 | 210193_s_at | proteolipid protein 1 (pelizaeus-merzbacher |
| | | disease, spastic paraplegia 2, uncomplicated) |
| 6 | 205611_at | tumor necrosis factor (ligand) |
| | | superfamily, member 12 |
| 7 | 206289_at | homeobox a4 |
| 8 | 218818_at | four and a half lim domains 3 |
| 9 | 202931_x_at | bridging integrator 1 |
| | 210201_x_at | |
| | 214439_x_at | |
| 10 | 210230_at | myelin protein zero (charcot-marie- |
| | | tooth neuropathy 1b) |
| 11 | 214023_x_at | tubulin, beta 2b |
| 12 | 210632_s_at | sarcoglycan, alpha (50 kda dystrophin- |
| | | associated glycoprotein) |
| 13 | 216894_x_at | cyclin-dependent kinase inhibitor 1c (p57, kip2) |
| 14 | 212457_at | transcription factor binding to ighm enhancer 3 |
| 15 | 213808_at | adam metallopeptidase domain 23 |
| 16 | 201431_s_at | dihydropyrimidinase-like 3 |
| 17 | 214122_at | pdz and lim domain 7 (enigma) |
| 18 | 215306_at | luteinizinz hormone/choriogonadotropin receptor |
| 19 | 202565_s_at | supervillin |
| 20 | 211964_at | collagen, type iv; alpha 2 |
| 21 | 205132_at | actin, alpha, cardiac muscle |
| 22 | 204628_s_at | integrin, beta 3 (platelet glycoprotein |
| | | iiia. antigen cd61) |
| | 204627_s_at | |
| 23 | 209169_at | glycoprotein m6b |
| 24 | 204736_s_at | chondroitin sulfate proteoglycan 4 |
| | | (melanoma-associated) |
| 25 | 204777_s_at | mal, t-cell differentiation protein |
| 26 | 209686_at | s100 calcium binding protein, beta (neural) |
| 27 | 216500_at | lectin, galactoside-binding, soluble. 1 (galectin 1) |
| 28 | 210319_x_at | msh homeobox homolog 2 (drosophila) |
| 29 | 212097_at | caveolin 1, caveolae protein, 22 kda |
| 30 | 206382_s_at | brain-derived neurotrophic factor |
| 31 | 204159_at | cyclin-dependent kinase inhibitor |
| | | 2c (p18, inhibits cdk4) |
| 32 | 204939_s_at | phospholamban |
| | 204940_at | |
| 33 | 202806_at | drebrin 1 |
| 34 | 204584_at | 11 cell adhesion molecule |

Cytoskeleton

| | | |
|---|---|---|
| 1 | 203389_at | kinesin family member 3c |
| 2 | 203151_at | microtubule-associated protein 1a |
| 3 | 202565_s_at | supervillin |
| 4 | 212565_at | serine/threonine kinase 38 like |
| 5 | 205132_at | actin, alpha, cardiac muscle |
| 6 | 205973_at | fasciculation and elongation |
| | | protein zeta 1 (zygin i) |
| 7 | 221246_x_at | tensin 1 |
| 8 | 218818_at | four and a half lim domains 3 |
| 9 | 209191_at | tubulin, beta 6 |
| 10 | 207876_s_at | filamin c, gamma (actin binding protein 280) |
| 11 | 202931_x_at | bridging integrator 1 |
| | 210201_x_at | |
| | 214439_x_at | |
| 12 | 214212_x_at | pleckstrin homology domain containing, |
| | | family c (with ferm domain) member 1 |
| 13 | 214023_x_at | tubulin, beta 2b |
| 14 | 213847_at | peripherin |
| 15 | 201841_s_at | heat shock 27 kda protein 1 |
| 16 | 210632_s_at | sarcoglycan, alpha (50 kda dystrophin- |
| | | associated glycoprotein) |
| 17 | 209651_at | tansforming growth factor beta |
| | | 1 induced transcript 1 |
| 18 | 202806_at | drebrin 1 |
| 19 | 214122_at | pdz and lim domain 7 (enigma) |

Cytoskeleton organization
and biogenesis

| | | |
|---|---|---|
| 1 | 203389_at | kinesin family member 3c |
| 2 | 209015_s_at | dnaj (hsp40) homolog, subfamily b, member 6 |
| 3 | 212793_at | dishevelled associated activator |
| | | of morphogenesis 2 |
| 4 | 202565_s_at | supervillin |
| 5 | 205132_at | actin, alpha, cardiac muscle |
| 6 | 218818_at | four and a half lim domains 3 |
| 7 | 209191_at | tubulin, beta 6 |
| 8 | 214023_x_at | tubulin, beta 2b |
| 9 | 214212_x_at | pleckstrin homology domain containing, |
| | | family c (with ferm domain) member 1 |
| 10 | 213847_at | peripherin |
| 11 | 214306_at | optic atrophy 1 (autosomal dominant) |
| 12 | 202806_at | drebrin 1 |
| 13 | 214122_at | pdz and lim domain 7 (enigma) |

Cell-substrate junction
assembly

| | | |
|---|---|---|
| 1 | 201383_at | integrin, alpha 5 (fibronectin receptor, |
| | | alpha polypeptide) |
| 2 | 221246_x_at | tensin 1 |
| 3 | 204628_s_at | integrin, beta 3 (platelet glycoprotein |
| | | iiia, antigen cd61) |
| | 204627_s_at | |

Actin cytoskeleton

| | | |
|---|---|---|
| 1 | 207876_s_at | filainin c, gamma (actin binding protein 280) |
| 2 | 202931_x_at | bridging integrator 1 |
| | 210201_x_at | |
| | 214439_x_at | |
| 3 | 214212_x_at | pleckstrin homology domain containing, |
| | | family c (with ferm domain) member 1 |
| 4 | 212565_at | serine/threonine kinase 38 like |
| 5 | 202565_s_at | supervillin |
| 6 | 205132_at | actin, alpha, cardiac muscle |
| 7 | 202806_at | drebrin 1 |
| 8 | 218818_at | four and a half lim domains 3 |
| 9 | 214122_at | pdz and lim domain 7 (enigma) |

Cyloplasm organization
and biogenesis

| | | |
|---|---|---|
| 1 | 210280_at | myelin protein zero (charcot- |
| | | marie-tooth neuropathy 1b) |
| 2 | 201389_at | integrin, alpha 5 (fibronectin receptor, |
| | | alpha polypeptide) |
| 3 | 221246_x_at | tensin 1 |
| 4 | 204628_s_at | imegrin, beta 3 (platelet glycoprotein |
| | | iiia: antigen cd61) |
| | 204627_s_at | |

Cell-substrate junction
assembly

| | | |
|---|---|---|
| 1 | 201389_at | integrin, alpha 5 (fibronectin receptor, |
| | | alpha polypeptide) |
| 2 | 221246_x_at | tensin 1 |
| 3 | 204628_s_at | integrin, beta 3 (platelet glycoprotein |
| | | iiia, antigen cd61) |
| | 204627_s_at | |

Actin cytoskeleton

| | | |
|---|---|---|
| 1 | 207876_s_at | filarnin c, gamma (actin binding protein 280) |
| 2 | 202931_x_at | bridging integrator 1 |
| | 210201_x_at | |
| | 214439_x_at | |
| 3 | 214212_x_at | pleckstrin homology domain containing, |
| | | family c (with ferm domain) member 1 |
| 4 | 212565_at | serine/threonine kinase 38 like |
| 5 | 202565_s_at | supervillin |
| 6 | 205132_at | actin, alpha, cardiac muscle |
| 7 | 202506_at | drebrin 1 |
| 8 | 218818_at | four and a half lim domains 3 |
| 9 | 214122_at | pdz and lim domain 7 (enigma) |

Cytoplasm organization
and biogenesis

| | | |
|---|---|---|
| 1 | 210280_at | myelin protein zero (charcot-marie- |
| | | tooth neuropathy 1b) |
| 2 | 201389_at | integrin, alpha 5 (fibronectin receptor, |
| | | alpha polypeptide) |
| 3 | 221246_x_at | tensin 1 |
| 4 | 204628_s_at | integrin, beta 3 (platelet glycoprotein |
| | | iiia, antigen cd61) |
| | 204627 s at | |

In another example, assessment of suspicious initial biopsies for expression of the classifier genes (done in this study by microarray) is carried out by any of a number of other gene quantification methods, which are well known and including those available for assessment of RNA in FFPE samples.

### Example 2

### Detection of Biomarkers Using Antibodies

Antibodies were screened against the protein products of the 114 stroma biomarker genes described in Example 1. 66 antibodies were identified that stain prostate tissue. Seventeen of these antibodies were tested using an immunofluorescence staining protocol for formalin fixed paraffin embedded (FFPE) tissues. The results are summarized in Table 7. Affinity purified antibodies produced by the HPA project (www.proteinatlas.org) were from Sigma Aldrich (see Table 7, column HPA Ab id) and used for IF labeling of prostate tissue for the biomarkers indicated (“Gene” column). Digital images were analyzed by CyteSeer histocytometry software (Vala Sciences, Inc.).

TABLE 7
Antibodies specific for stroma biomarkers

| | | FFPET-IF | | | | |
|---|---|---|---|---|---|---|
| Gene | HPA_Ab id | tested | Category | PTT | Ab Species | Clone |
| AMACR | CAB001809 | yes | R | N/A | Mouse | 13H4 |
| FOLH1 | HPA010593 | yes | R | N/A | Rabbit | N/A |
| KRT19 | CAB000031 | yes | R | N/A | Mouse | RCK108 |
| ACTA2 | CAB000002 | yes | R | TBD | Mouse | 1A4 |
| DES | CAB000034 | yes | R | TBD | Mouse | D33 |
| VIM | CAB000080 | yes | R | TBD | Mouse | V9 |
| COL4A2 | CAB010751 | yes | DC | IF confirmed | Mouse | COL-94 |
| HSPB8 | HPA015876 | yes | DC | IF confirmed | Rabbit | N/A |
| PDLIM7 | HPA018794 | yes | DC | IF confirmed | Rabbit | N/A |
| ALDH3A2 | CAB020692 | no | DC* | IHC | Rabbit | N/A |
| | | | | confirmed | | |
| FBN1 | CAB002670 | no | DC* | IHC | Mouse | 11C1.3 |
| | | | | confirmed | | |
| CAV1 | CAB003791 | yes | DC | TBD | Mouse | E249 |
| DMPK | HPA007164 | yes | DC | TBD | Rabbit | N/A |
| DPYSL3 | HPA010948 | yes | DC | TBD | Rabbit | N/A |
| KCTD17 | HPA018549 | yes | DC | TBD | Rabbit | N/A |
| SVIL | HPA020138 | yes | DC | TBD | Rabbit | N/A |
| CRTAC1 | HPA008175 | no | DC | TBD | Rabbit | N/A |

*= no yet confirmed by IF;
R = internal control reference candidate gene;
DC = diagnostic candidate;
PTT = Evidence that signal intensity appears dependent on “Proximity to Tumor.”

Histocytometric analysis was performed on whole slide images stained with stroma markers identified at the RNA level as exhibiting a gradient of expression based on “proximity to tumor” (PTT).

FIG. 7 shows the result obtained by staining sections with anti-COL4A2 (collagen, type IV, alpha 2, Alexa Fluor® 594) and anti-PDLIM7 (PDZ and LIM domain 7 (enigma), Alexa Fluor® 488). Nuclei are labeled with DAPI. The slide was scanned as 20× but shown here at low resolution. As shown in FIG. 7B, stroma cells within the infiltrating tumor labeled with the anti-COL4A2 antibody but not with the anti-PDLIM7 antibody. Stroma remote from tumor (bottom right and bottom center panel) readily exhibit both COL4A2 and PDLIM7 staining. FIG. 7B shows an example of two antibody biomarkers PDLIM and COL4A2, demonstrating tumor proximity-dependent fluorescent signal intensity.

As shown in FIG. 7, the observed response of RNAs in stroma to the presence of cancer nearby is reiterated at the protein level for the indicated biomarkers. For COL4A2 and PDLIM7, it appears that tumor suppresses expression, and expression reaches “normal” stroma levels at a distance of about 8 mm and 3-5 mm from the tumor, respectively.

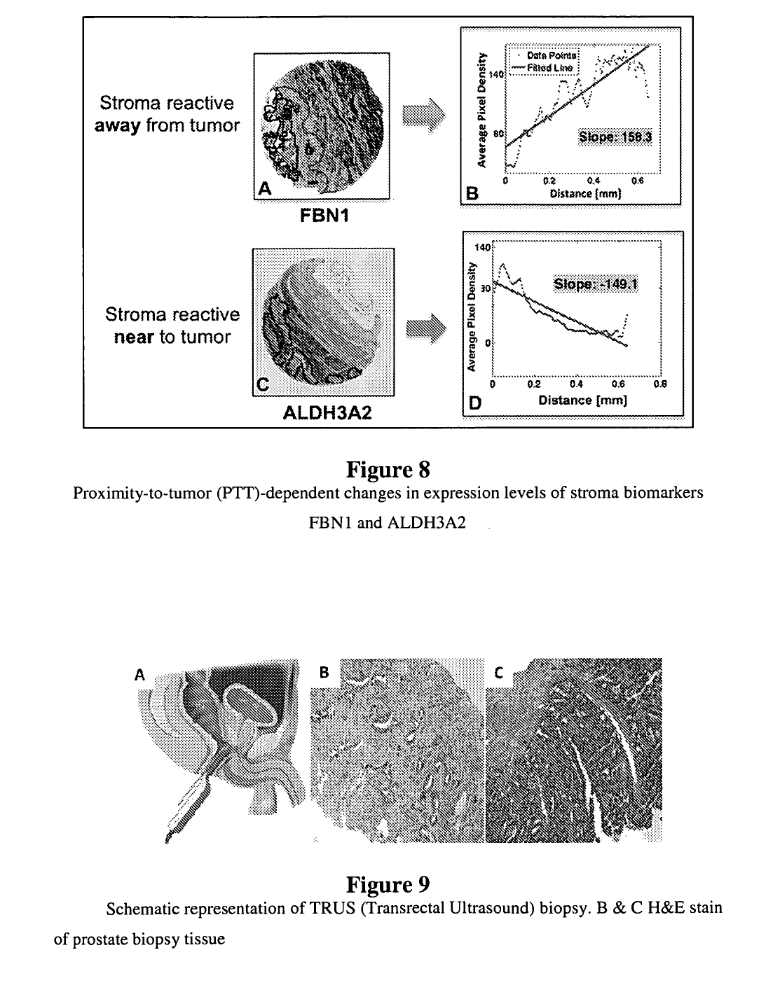

FIG. 8 shows results obtained with two additional biomarkers exhibiting PTT behavior (ALDH3A2 (aldehyde dehydrogenase 3 family, member A2) and FBN1 (fibrillin)). In this study, Human Protein Atlas (HPA) images were obtained following immunohistochemically labelling using daiminobenzidine (see FIG. 8A, 8C). Gradients were observed visually (FIG. 8A, 8C) and also analyzed automatically using a custom imaging algorithm. Panels 8B and 8D were generated by plotting pixel intensity differences at fixed distances from tumor (contour lines in panels A and C, in the tissue microarray cores, which were approximately 1 mm in diameter). In this study, because the automated histocytochemistry was used to analyze IHC images of TMA cores (limited size of ˜1 mm), the PTT effect could be observed only for a short distance. As shown in FIG. 8, ALDH3A2 expression was observed to be higher closer to tumor, in contrast to the other three candidate tumor-responsive stromal biomarkers where expression was lower near tumor. Thus, FIG. 8 shows stroma markers exhibiting reactivity away from tumor (B) and near to tumor (C), plotted as increasing and decreasing slopes, respectively. These data demonstrate successful mining of the HPA database for stroma biomarkers for use in the provided methods.

In another example, immunofluorexcent (IF) whole slide images for ALDH3A2 and FBN1 are obtained as described herein to determine the overall staining behavior for these markers.

### Example 3

### Algorithmic Determination of Tumor-Proximity-Dependent Signal Intensity of Stroma Markers Using HPA Images

An algorithmic determination of tumor-proximity-dependent signal intensity of stroma markers was performed on HPA images. The stroma mask was identified. The edges of what was excluded were assumed to mark the gland or cancer mask. The distance of each pixel in the stroma mask from all gland/cancer in the image then was determined by finding the Euclidian distance of each pixel from all points in the outline of the gland/cancer mask as identified above. This operation resulted in a contour map that assigned a distance to each stroma pixel. The stroma pixels then were put into bins (contour levels) depending on their distance value. In FIGS. 8A and 8C, black lines at the left-hand side of the ˜1 mm diameter cores represent the identified gland/cancer mask; contour levels were marked in the stromal space. The average pixel density then was calculated for each bin and plotted versus distance. The slope of a linear fit was then used as the measure of how the intensity of the signal for each biomarker (obtained by antibody staining) changed as a function of distance from the gland/cancer; panels B and D show average pixel density versus distance from the gland/cancer mask, determined based on the images in panels A and C, respectively. As shown in FIGS. 8A and 8B, staining signal (detected expression levels) for FBN1 increased with the distance; this characteristic was quantified accordingly by the positive slope value of 158.3. As shown in FIGS. 8C and 8D, staining signal (detected expression levels) for ADH3A2 decreased with distance from tumor, which characteristic was quantified by the negative slope of −149.1.

The present invention is not to be limited in scope by the embodiments disclosed herein, which are intended as single illustrations of individual aspects of the invention, and any that are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### Example 4

### Differential Tissue Staining Patterns of SVIL in Prostate Stroma Versus Tumor Epithelial Structures

Formalin fixed, paraffin embedded 5 μm tissue sections were stained with anti-SVIL antibodies and visualized using an Alexa Fluor® 488 labeled secondary antibody. Staining was recorded using a 3DHISTECH digital microscopic scanner. Homogeneous cellular staining was observed in stroma tissue characterized by myocyte structures. Of note is the even staining pattern with no discernible staining differences of subcellular structures. Tumor epithelial tissue surrounding glandular structures showed markedly different staining patterns: a stippled staining pattern was observed suggesting that SVIL may be located in cytoplasmic vesicular structures. Staining was not observed in normal appearing epithelial/glandular structures. The differential expression may further be indicative of the different subforms of SVIL being differentially expressed in the subtypes of prostate tissue structures examined whereby the antibodies used do not distinguish the subtypes. These findings suggest that SVIL may not only be a prostate cancer stromal marker but may also distinguish normal from cancerous epithelial tissue and may thus be of diagnostic value either as a single biomarker or in conjunction with others biomarkers.

1. A diagnostic method, comprising:
a) contacting a test biological sample from a patient with an agent that specifically binds to a prostate stroma biomarker; and
b) determining an expression level of the biomarker in the test biological sample,
wherein the method detects the presence of a prostate lesion or growth-dysregulated cell in the patient and the test biological sample is a non-tumor sample or is essentially tumor-free.

2. A diagnostic method, comprising:
a) contacting a test sample with an agent that specifically binds to a prostate stroma biomarker, wherein the test sample is from a biopsy needle core from a prostate;
b) detecting an amount of binding of the agent to the sample, thereby indicating an expression level of the prostate stroma biomarker,
wherein the method is capable of detecting the presence of a prostate tumor with a percent volume coverage of greater than 0 1, 2, 3, 4, 5, 10, 15, 20, 25, 30, 35, 40, 45, 50, 55, 60, 65, 70, 75, 80, 85, 90, 95, 96, 97, 98, 99%.

3. A diagnostic method, comprising:
a) contacting a test sample with an agent that specifically binds to a prostate stroma biomarker, wherein the test sample is from a biopsy needle core from a prostate; and
b) detecting an amount of binding of the agent to the sample, thereby indicating an expression level of the prostate stroma biomarker,
wherein the prostate stroma biomarker exhibits proximity-to-tumor dependent expression at a distance of at least a 1, 2, 3, 4, 5, 6, 7, or 8 mm.

4. A diagnostic method, comprising:
a) contacting a test sample with an agent that specifically binds to a prostate stroma biomarker, wherein the test sample is from a biopsy needle core from a prostate; and
b) detecting an amount of binding of the agent to the sample, thereby indicating an expression level of the prostate stroma biomarker,
wherein the prostate stroma biomarker exhibits a stromal signal of at least a 1, 2, 3, 4, 5, 6, 7, or 8 mm.

5. A method for detecting a lesion or growth dysregulated cell in a prostate, comprising:
a) contacting a test sample with an agent that specifically binds to a prostate stroma biomarker, wherein the test sample is from a biopsy needle core from a prostate;
b) determining an amount of binding of the agent to each of a plurality of locations within the sample; and
c) determining a three-dimensional position of the lesion or growth dysregulated cell in the prostate, based on the amounts so determined.

6. A method for localizing a growth dysregulated cell in a prostate, comprising:
a) obtaining one or more prostate biopsy samples from a subject;
b) constructing a sample map, wherein the one or more prostate biopsy sample is mapped to the subject's prostate;
c) determining an expression level of a prostate stromal biomarker in the sample; and
d) plotting the expression level to the sample map, thereby determining the location of the growth-dysregulated cell.

7. The method of any of claims 1-6, wherein the prostate stroma biomarker is selected from the group consisting of ALDH3A2, PDLIM7 COL4A2, HSPB8, and FBN1 gene products.

8. The method of any of claims 1-6 wherein the prostate stroma biomarker is selected from the group consisting of COL4A2, HSPB8, PDLIM7, ALDH3A2, FBN1, CAV1, DMPK, DPYSL3, KCTD17, SVIL, and CRTAC1 gene products.

9. The method of any of claims 1-6, wherein the prostate stroma biomarker is selected from the group consisting of products of the genes listed in Table 3.

10. The method of any of claims 1-4, wherein the agent is an antibody or fragment thereof.

11. The method of claim 10, wherein the antibody is labeled with a detectable marker.

12. The method of any of claims 1-4, wherein the agent is a polynucleotide.

13. The method of any of claims 2-12, wherein the sample is a non-tumor bearing sample.

14. The method of any of claims 1-6, wherein the prostate stroma biomarker comprises a plurality of prostate stroma biomarkers.

15. The method of any of claims 1-14, wherein the sample comprises a fixed prostate tissue sample.

16. The method of any of claims 1-15, wherein the expression level is determined for a location within the prostate stroma.

17. The method of claim 16, wherein the location is tumor-adjacent, tumor-close, or tumor-near.

18. The method of claim 16, wherein the location is within 2, 3, 4, 5, 6, 7, 8, 9, 10, 11, 12, 13, 14, or 15 mm of a prostate tumor.

19. The method of any of claims 1-18, wherein the prostate stroma biomarker exhibits increased expression with increased proximity to a prostate tumor.

20. The method of any of claims 1-18, wherein the prostate stroma biomarker exhibits decreased expression with increased proximity to a prostate tumor.

21. The method of any of claims 1-18, wherein the method detects increased expression levels of the prostate stroma biomarker with increased proximity to a prostate tumor.

22. The method of any of claims 1-18, wherein the method detects decreased expression levels of the prostate stroma biomarker with increased proximity to a prostate tumor.

23. The method of any of claims 1-18, wherein the method detects a prostate tumor with at least 80, 85, 90, 95, 96, 97, 98, 99, or 100% accuracy.

24. The method of any of claims 1-18, wherein the method detects an early-stage prostate cancer.

25. The method of any of claims 1-24, wherein the biomarker includes a FBN1 gene product.

26. The method of any of claims 1-25, wherein the biomarker includes an ALDH3A2 gene product.

27. The method of any of claims 1-24, wherein the biomarker includes a COL4A2 gene product.

28. The method of any of claims 1-27, wherein the biomarker includes a PDLIM7 gene product.

29. The method of any of claims 1-28, wherein the sample comprises fresh prostate tissue sample.

30. The method of any of claims 1-29, wherein the sample comprises a frozen prostate tissue sample.
